(19) United States
(12) Patent Application Publication
(10) Pub. No.: US 2023/0099367 A1
(43) Pub. Date: Mar. 30, 2023

(54) PURMORPHAMINE AS A SMALL COMPOUND POSITIVE ALLOSTERIC MODULATOR OF SECRETIN RECEPTOR FOR THE TREATMENT OF HYPERTENSION

(71) Applicant: VERSITECH LIMITED, Hong Kong (CN)

(72) Inventors: Kailash SINGH, Hong Kong (CN); Billy Kwok Chong CHOW, Hong Kong (CN)

(21) Appl. No.: 17/617,895

(22) PCT Filed: Apr. 15, 2020
(86) PCT No.: PCT/CN2020/084879

§ 371 Date: Dec. 9, 2021

Related U.S. Application Data
(60) Provisional application No. 62/834,663, filed on Apr. 16, 2019.

(51) Int. Cl. A61K 31/5377; A61P 9/12

(52) U.S. Cl. CPC A61K 31/5377; A61P 9/12

(57) ABSTRACT

The subject invention pertains to methods and compositions for treating hypertension. In specific embodiments, the methods comprise administering KSD179019 to a subject in need of hypertension treatment. In further embodiments, the methods comprise administering a KSD179019 analog or derivative to a subject in need of hypertension treatment. Also provided are methods to design and manufacture KSD179019 analogs and derivatives with enhanced and prolonged anti-hypertensive therapeutic efficacy.

### CROSS-REFERENCE TO RELATED APPLICATIONS

This application is the U.S. national stage application of International Patent Application No. PCT/CN2020/084879, filed Apr. 15, 2020, which claims the benefit of U.S. Provisional Application Ser. No. 62/834,663, filed Apr. 16, 2019, each of which are hereby incorporated by reference in its entirety including any tables, figures, or drawings.

### BACKGROUND OF THE INVENTION

Hypertension is a pathological condition with persistently elevated blood pressure (i.e. >140/90 mmHg) and is acknowledged as an important risk factor for heart and cerebrovascular diseases. Persistently elevated blood pressure is clinically proven to increase the risk of potentially life-threatening disease such as heart disease, heart attack, strokes, heart failure, peripheral arterial diseases, aortic aneurysms, kidney disease, and vascular dementia etc. Chronically elevated blood pressure with no other serious pathological ailments is known as primary hypertension. Secondary hypertension is identified as chronically increased blood pressure which is caused by an underlying disorder. In some cases, due to the underlying pathological condition in secondary hypertension, conventional drugs are not able to affect the blood pressure of the patient. When patients are non-responsive to mono-therapy or a combination of three different conventional drugs, hypertension is known as resistant hypertension [1]. According to the 7th report of joint national committee (JNC7), systolic blood pressure <140 mmHg and diastolic blood pressure of <90 mmHg is recommended as a goal for patients with primary hypertension. For secondary hypertension patients, a goal of <130 mmHg systolic pressure with <80 mmHg diastolic pressure is recommended. However, only 50% of patients suffering from secondary hypertension are reported to reach the advised levels. Currently, a wide range of drugs are available for the treatment of hypertension. Despite the wide range of anti-hypertensive drugs, a large portion of patients remains resistant to the conventional drugs available. Therefore, there is a need for new treatments for hypertension. The global market size of hypertension is estimated to reach 9.9 billion dollars by the year 2025 [2]. China is documented to have a growing number of hypertensive patients every year. In the year 2005-2009, it was shown that 42% of Chinese adults aged 35-70 years were hypertensive and blood pressure of only 8.2% of hypertensive patients was controlled. In 2014, the total number of premature deaths relating to hypertension in China was 2 million and the corresponding medical cost was 3.6 billion [3]. Further, 70% of heart strokes and 50% of myocardial infarctions were attributed to hypertension in China [4].

The inventors advantageously uncovered the roles of secretin and its receptor, secretin receptor (SCTR), in water/salt homeostasis and blood pressure [5-11]. Using a secretin gene knockout mouse model, it was discovered that mice lacking secretin expression suffered from hypertension and intravenous or intraperitoneal administration of secretin resulted in an acute reduction in blood pressure. However, secretin has a short half-life when administered to a subject. Therefore, the inventors discovered a small compound agonist of SCTR that is capable of modulating SCTR activity and has a prolonged biological effect and serum half-life.

### BRIEF SUMMARY OF THE INVENTION

Provided are compositions and novel uses of the small molecule puromorphamine (i.e. KSD179019) that functions as a positive allosteric modulator (PAM) of the SCTR for the treatment of hypertension. The methods and compositions of the instant invention provide a novel use for KSD179019. Without wanting to be bound by theory it is suggested that the efficacy of KSD179019 in providing a sustainable reduction in blood pressure is based on KSD179019's ability to maximize the response of secretin at the SCTR. Due to its positive allosteric effects, KSD179019 provides targeted and sustained effects while reducing the potential for off-target effects and unwanted clinical side effects.

Further provided are methods and systems for designing KSD179019 analogs and derivatives with enhanced anti-hypertensive properties based on providing prolonged secretin-SCTR interactions and enhancing the specificity of KSD179019 for the transmembrane SCTR region.

### BRIEF DESCRIPTION OF THE DRAWINGS

FIG. 1 shows a pharmacophore model developed from the secretin binding site at the SCTR. A). 3D interaction diagram of SCTR (white) in complex with secretin peptide (red) with selected residues for pharmacophore design highlighted in blue color. B). 3D pharmacophore model developed from the secretin binding site on the SCTR. The residues selected to function as a pharmacophore model are Glu26, Glu30, Pro55, Arg61, Val65, and Arg69.

FIG. 2 shows pharmacophore-based hits from the secretin binding site at the SCTR. A). Details of eight pharmacophores with their 3D coordinates on the six amino acid residues of SCTR, Glu26, Glu30, Pro55, Arg61, Val65, and Arg69. B). 3D model of SCTR (gray) with eight selected residues for pharmacophore design highlighted—Glu26 as hydrogen donor and acceptor, Glu30 as hydrogen donor and hydrogen acceptor, Pro55 as aromatic, Arg61 as a hydrogen donor, Val65 as hydrophobic pharmacophore and Agr69 as hydrogen donor. C). List of pharmacophore based hits as screened from the PubChem database showing 23 out of 23 hits.

FIG. 3 shows a screening for the allosteric site of the human secretin receptor (SCTR). A). Full human SCTR receptor model in complex with WDN (Green), cWDN (Red) and SCT. B). Top view of SCTR complex with WDN and cWDN providing insight into the active site of the complex with all three ligands C). 2D interaction of cWDN showing the amino acid residue's nitration with receptor Tyr146, Asn192, Lys195, Asp196, Trp286, Leu199, Leu370, and Phe222.

FIG. 4 shows pharmacophore-based hits using cWDN bound to the secretin receptor (SCTR). A). Details of the pharmacophore and the 3D coordinates at cWDN's tryptophan and aspartic acid residues. B). 3D model of SCTR (gray) and cWDN (yellow) with selected residues for four pharmacophores—two aromatic pharmacophores on tryptophan, a hydrogen bond donor and a hydrogen acceptor on the alcohol group of aspartic acid residues and one hydrogen bond acceptor pharmacophore at the oxygen atom of aspartic acid. C). A representative list of pharmacophore based hits as screened from the PubChem database showing 27 out of 71,630 hits.

FIG. 5 shows in vitro agonistic effects of selected compounds after in silico based screening. Twenty-one compounds (100 μM concentration) were tested for a cAMP response to human SCTR overexpressed in CHO cells with the secretin peptide (SCT) at 1 μM concentration. KSD179019 as a potential agonist with a slight activation of the SCTR was selected for future dose-dependent analyses. (* p<0.05).

FIG. 6 shows in vitro antagonistic effects of selected compounds after in silico based screening. Twenty-one compounds (10 μM concentration) together with 1 μM of the secretin peptide (SCT) were tested for a cAMP response to human SCTR overexpressed in CHO cells. KSD179019 as a potential agonist with slight activation of the SCTR was selected for future dose-dependent analyses. Additionally, H1810, BMS2299 and SAR2370 displayed potential antagonistic effects and were also selected for further analyses. (* p<0.05).

FIG. 7 shows the basic structure of KSD179019.

FIG. 8 shows an in vitro analysis of human secretin receptor (SCTR) with selected compounds. In vitro cAMP assay in CHO cells overexpressing a human SCTR. Y-axis representing the fold change in cAMP response in the CHO cells transfected with a human SCTR after being activated by different concentrations of ligands displayed on the X-axis. Secretin (SCT), KSD179019 and secretin at different concentration plus KSD179019 at 100 μM concentration. (n=3).

FIG. 9 shows an ex vivo cAMP assay performed on different isolated tissues treated with KSD179019. cAMP response in isolated organs from mice (C57) stimulated with secretin (1 μM), KSD179019 (100 μM) and secretin (1 μM) plus KSD179019 (100 μM). A). Isolated Duodenum B). Isolated pancreas. C). Isolated mouse cerebellum. D). Isolated mouse kidney as the control since secretin peptide itself was not able to produce a cAMP response. (n=2-8/group; * p<0.05; ** p<0.01, ****p<0.001).

FIG. 10 shows an effect of KSD179019 upon intracerebroventricular injection on plasma vasopressin levels in C57 mice. Plasma vasopressin (vp) concentration over time after i.c.v injection of SCT, KSD179019, and KSD179019 plus secretin; ACSF is a vehicle control. The result shows a statistically significant increase in the plasma vasopressin levels after stimulation by KSD179019 and secretin (SCT); SCT as the positive control, ACSF as a negative control. (n=6/group; * p<0.05; ** p<0.01, ****p<0.001).

FIG. 11 shows an effect of secretin upon intraperitoneal administration in C57 mice. A). The tracings show systolic and diastolic blood pressure (BP) before and after intraperitoneal injection of PBS and SCT. B). Systolic and diastolic BP dropped at 5 min after secretin injection, the reduction was statistically significant compared with time 0 BP (initial BP). No BP drop at 5 min after PBS injection compared with time 0 BP (initial BP). (n=6/group; * p<0.05; ** p<0.01).

FIG. 12 shows an effect of KSD179019 upon intravenous administration in spontaneously hypertensive rats. Tail vein administration of KSD179019 in spontaneously hypertensive rats (SHR) results in a statically significant drop in blood pressure 20 hours post-injection. A). Administration of KSD179019 at 0.26 μg/g body weight (BW). B). Administration of saline as a vehicle control. C). Bar graph representation of systolic and diastolic blood pressure before injection, 20 hours and 60 hours post KSD179019 injection. The blood pressure at 20 hours post-injection is calculated to be significantly different. (n=6/group; * p<0.05; ** p<0.01).

FIG. 13 shows tail vein administration of KSD179019 and oral administration of a combination of Azilsartan (Az) plus Chlorthalidone (CLT) in spontaneously hypertensive rats. A). Representative image of one rat with the percentage mean arterial blood pressure (% MABP) represented on the Y-axis and time in hours on the x-axis. B). Bar graph representation showing that the combination therapy was able to reduce the MABP by about 20% at 20 hours post oral administration but did not show any significant reduction thereafter. In contrast, KSD179019 reduced the MABP by 10% at 40 and 60 hours post-intra-venous administration demonstrating the ability of KSD179019 to provide sustained blood pressure reduction in vivo (N=2).

FIG. 14 shown the systolic, diastolic and mean arterial pressures were significantly increased in SCT−/− mice compared with 6-month-old C57BL/6N mice (n=12/group; *, p<0.05)

FIG. 15 shows an in vitro assay demonstrating that KSD179019 is capable of producing positive allosteric effect. The maximal cAMP production by secretin (SCT) was observed at 1 μM concentration. When KSD179019 (KSD) was added along with SCT, the combination displayed an enhanced efficacy of 131.9% of maximal cAMP and a shift of EC50 from 56 nM for SCT alone to 13 nM when in combination with KSD (100 μM).

FIG. 16 shows the hypotensive action of the secretin receptor small compound allosteric agonist KSD179019 on spontaneously hypertensive rat (SHR) following tail-vein administration of KS0179019 (0.26 μg/g body weight). Statistically significant drops in diastolic and systolic blood pressure from 20 to 50 hours post injection was observed.

FIG. 17 shows the hypotensive action of the secretin receptor small compound allosteric agonist KSD179019 on spontaneously hypertensive rat (SHR) following oral administration of KSD179019 (30 mg/kg body weight (BW), 50 mg/kg BW and 80 mg/kg BW) on SHR. Statistically significant drops in diastolic and systolic blood pressure from 20 to 50 hours post injection were observed.

FIG. 18 shows the analyses of organ and cell specific toxicity of KSD179019 in vitro using a MTT reagent cell proliferation assay. KSD179019 was observed to have a) No hepatotoxicity (i.e. no cell death in HepG2cells) as well as b) No nephrotoxicity (i.e. no cell death in Hek293 cells). (* p<0.05; ** p<0.01).

FIG. 19 shows the analyses of KSD179019 was tested for its carcinogenic potential using an Ames test for mutagenicity. The Ames test was performed using different salmonella strains a). TA98, b). TA100, c). TA1535 and d.) TA1537 and e). a combination of two E. Coli strains (uvrA and pKM101). (* p<0.05; ** p<0.01).

FIG. 20 shows the analyses of KSD179019 for its carcinogenic potential using an Ames test for mutagenicity. The Ames test was performed using different salmonella strains in the presence and absence of S9 liver extract a). TA98, b). TA100, c). TA1535 and d.) TA1537 and e). a combination of two E. Coli strains (uvrA and pKM101). (* p<0.05; ** p<0.01).

FIG. 21 shows an acute study over 14 days to estimate the lethal dose 50 (LD50) in males, i.e., the dose which is lethal for 50 percent of male animals treated. The male C57BL/6J mice were orally administered with 2000 mg/kg BW of KS0179019 and were observed for 14 days continually with no observed adverse effects. Specifically, a) there was no significant change (above 2 grams) in body weight of individual mice and b) no statistically significant change in the body temperature. (* p<0.05; ** p<0.01).

FIG. 22 shows an acute study over 14 days to estimate the lethal dose 50 (LD50) in females, i.e., the dose which is lethal for 50 percent of female animals treated. The female CS7BL/6J mice were orally administered with 2000 mg/kg BW of KS0179019 and were observed for 14 days continually with no observed adverse effects. Specifically a) there was no significant change (above 2 grams) in body weight of individual mice and b) no statistically significant change in the body temperature. (* p<0.05; ** p<0.01).

FIG. 23 shows an acute study over 14 days to estimate the oral LD50. Female C57BL/6J mice were orally administered with 5000 mg/kg BW of KSD179019 and were observed for 14 days continually with no observed adverse effects. Specifically, a) there was no significant change (above 2 grams) in body weight of individual mice and b) no statistically significant change in the body temperature. (* p<0.05; ** p<0.01).

FIG. 24 shows the standard calibration curve developed by the HPLC grade KSD179019 on LCMS/MS-Q TOF. The table represents the calculation of standard concentration with its corresponding peak area. The graph represents the standard straight line in the graph with R2 value of 0.9999 with the straight line equation of y=323.58x+2643.9.

FIG. 25 shows a screening for other positive allosteric modulators based on the structure of KSD179019 after designing via structure aided drug design (SADD). Nine derivatives of KSD179019 (KSDd_1, KSDd_2, KSDd_3, KSDd_4, KSDd_5, KSDd_6, KSDd_7, KSDd_8 and KSDd_9 were developed by custom chemical synthesis and then tested in an in vitro receptor activation assay. The preliminary screening assay revealed KSDd_4, KSDd_7, KSDd_8 and KSDd_9 as potential positive allosteric agonists.

FIG. 26 shows the dose dependent effect of KSDd_4, KSDd_7, KSDd_8 and KSDd_9 as potential positive allosteric agonists in a Secretin Receptor activation assay. It was observed that a. KSDd_4, b. KSDd_7, c) KSDd_8 and d) KSDd_9 were able to potentiate secretin peptide in the activation of the secretin receptor.

FIG. 27 shows an in vitro comparative analysis of KS0179019 derivatives (i.e. KSDd_4, KSDd_7, KSDd_8 and KSDd_9) with KSD179019. It was observed that KSDd_7 and KSDd_8 were the most potent derivative followed by KSDd_4 and KSDd_9.

### DETAILED DISCLOSURE OF THE INVENTION

Provided are compositions and novel methods of use of the small molecule KSD179019. It has been discovered that the SCTR is a novel target for hypertension therapy and that secretin interaction with the SCTR effectuates a reduction in blood pressure. Advantageously, KSD179019 has been identified as a positive allosteric agonist of the SCTR affecting blood pressure and other pathophysiological processes. Because the secretin peptide and synthetic secretin analogs have a short half-life in the circulation after in vivo administration and a sustainable activation of the SCTR is needed to achieve pathophysiological effects, for example, clinically relevant, prolonged effects on blood pressure.

In embodiments of the instant invention, KSD179019 and compositions comprising a therapeutically effective amount of KSD179019 are provided for the effective and sustained reduction of blood pressure in a subject in need of blood pressure reduction. Interaction of KSD179019 with the SCTR provides a positive allosteric effect for sustained secretin binding to the SCTR and sustained SCTR signaling resulting in persistent effects on blood pressure.

In preferred embodiments of the invention, methods and compositions are provided for treating a subject suffering from hypertension, which methods comprise administering to said subject compositions comprising KSD179019 and a pharmaceutically acceptable carrier or excipient to the subject.

In further preferred embodiments of the invention, methods and compositions are provided for treating a subject suffering from hypertension, which methods comprise administering compositions comprising a KSD179019 analog and/or derivative and a pharmaceutically acceptable carrier or excipient to the subject.

The term “normal” blood pressure as used herein refers to a systemic systolic pressure of 120 mmHg and systemic diastolic blood pressure of 80 mmHg. The term “pre-clinical” hypertension as used herein refers to a systemic systolic blood pressure between 120 and 140 mmHg and systemic diastolic blood pressure between 80 and 90 mmHg. The term “hypertension” as used herein refers to systemic systolic blood pressure above 140 mmHg and systemic diastolic blood pressure above 90 mmHg.

In some embodiments, the methods of the invention treat hypertension that is primary hypertension, e.g., when high blood pressure occurs without identification of an underlying disease.

In other embodiments, the methods of the invention treat hypertension that is secondary hypertension, e.g., hypertension caused by an underlying disease and/or condition including, but not limited to, chronic kidney disease, e.g., diabetic nephropathy, polycystic kidney disease, glomerular disease, and renovascular stenosis; sleep apnea; a dysfunction of the adrenal gland including, but not limited to, pheochromocytoma and aldosteronism; Cushing's syndrome; thyroid dysfunction; coarctation of the aorta; obesity; pregnancy; and/or medications and supplements including, but not limited to, pain relievers, birth control pills, antidepressants, and immunosuppressants.

In yet further embodiments, the methods of the invention treat hypertension that is resistant hypertension. Resistant hypertension is hypertension that is not affected by administration of monotherapy or combination therapy using three or more conventional anti-hypertensive drugs. Without wanting to be bound by theory, the instant invention identifies KSD179019 as a positive allosteric agonist of the SCTR and provides methods using KSD179019 and/or KSD179019 analogs and/or KSD179019 derivatives that affect multiple pathways related to SCTR, which multiple pathways reduce blood pressure and treat hypertension that has previously been therapy resistant. In specific embodiments, the methods provided comprise administering a composition of the instant invention to a subject suffering from hypertension, wherein the composition comprises a therapeutically effective amount of KSD179019 and/or KSD179019 analog and/or derivative and a pharmaceutically acceptable carrier.

The therapeutically effective amount is an amount of the composition of the invention, which results in a reduction of a subject's systemic blood pressure to a level that is either normal blood pressure or blood pressure that is as close to normal blood pressure as clinically possible without inducing symptoms of hypoperfusion in the subject

The rate or time period over which the blood pressure is reduced to the target pressure is determined by the clinician based on the needs of the individual subject. For example, excessively high blood pressure may need to be reduced fast to a blood pressure level that is either normal blood pressure or as close to normal blood pressure as obtainable without causing hypoperfusion.

In further embodiments, the methods of the invention comprise administering at least one fast-acting anti-hypertensive agent to achieve a fast reduction in blood pressure to the desired blood pressure level followed by administering a composition of the instant invention to maintain the blood pressure at a normal level or as close to a normal level as obtainable without causing hypoperfusion. The fast-acting anti-hypertensive agent can be any agent that is conventionally used to lower blood pressure over a short period of time and can be administered systemically, e.g., by intravenous infusion.

In some embodiments, a chronically elevated blood pressure may need to be reduced gradually in a subject at risk for hypoperfusion due, e.g., to co-morbidities and the method of the invention comprises administering a composition of the invention comprising KSD179019 and/or a KSD179019 analog and/or derivative and a pharmaceutically acceptable carrier in an incremental manner increasing the amount of the composition based on its effect on the blood pressure up to an amount that maintains a target blood pressure. It is within the purview of the ordinarily skilled clinician to adjust the target blood pressure to the individual subject. A subject at risk for hypoperfusion due to co-morbidities may suffer from any disease and/or condition causing a reduction in organ or tissue perfusion and include, but not limited to, atherosclerosis, cardiovascular disease, arterial stenosis, left ventricular heart failure. Symptoms of hypoperfusion as used herein are known to the ordinarily skilled clinician and include any symptom that a vital organ does not receive sufficient blood supply, which symptoms include, but are not limited to, pain, dizziness, double vision, tinnitus, muscle spasm, gastrointestinal pain, diarrhea, and elevation of blood values indicating organ damage including liver, kidney, gastrointestinal tract, muscle cardiac and/or cerebral damage.

For example, a target blood pressure in a subject at risk for hypoperfusion can be a blood pressure with systolic levels between 120 and 140 mmHg and diastolic levels between 80 and 90 mmHg, where the subject experiences hypoperfusion symptoms at lower blood pressure levels. Accordingly, the ordinary skilled clinician can adjust the therapeutically effective amount of a composition of the instant invention to achieve a satisfactory reduction in blood pressure while avoiding the occurrence of symptoms caused by hypoperfusion.

In some embodiments, methods and compositions are provided for treating a subject suffering from pre-clinical hypertension, which methods comprise administering compositions comprising KSD179019 and/or a KSD179019 analog and/or derivative and a pharmaceutically acceptable carrier to the subject.

In other embodiments, methods and compositions are provided for treating a subject suffering from primary hypertension, which methods comprise administering compositions comprising KSD179019 and/or a KSD179019 analog and/or derivative and a pharmaceutically acceptable carrier to the subject.

In yet other embodiments, methods, and compositions are provided for treating a subject suffering from secondary and/or resistant hypertension, which methods comprise administering compositions comprising KSD179019 and/or a KSD179019 analog and/or derivative and a pharmaceutically acceptable carrier to the subject.

The methods further comprise treating the disease and/or condition underlying secondary hypertension and administering a composition of the instant invention comprising KSD179019 and/or a KSD179019 analog and/or derivative and a pharmaceutically acceptable carrier at an amount that is adjusted such that the amount of the composition is reduced according to the extent to which the underlying disease and/or condition is resolved and/or treated and either a normal blood pressure or a blood pressure as close to a normal blood pressure as obtainable without causing hypoperfusion is achieved.

The methods of the instant invention also comprise administering KSD179019 and/or a KSD179019 analog and/or derivative alone or in combination with at least one further blood pressure reducing agent. The compositions of the instant invention comprising KSD179019 and/or a KSD179019 analog and/or derivative and at least one or more anti-hypertensive agent allow sufficient control of a subject's blood pressure while reducing the levels of the individual compounds administered in such combination therapy. The combination compositions of the instant invention result in a therapeutically effective reduction of the blood pressure in a subject while keeping at minimum unwanted side effects often encountered when single agents at high doses to control blood pressure.

In some embodiments, the composition of the invention comprising KSD179019 and/or a KSD179019 analog and/or derivative and at least one additional anti-hypertensive agent are each administered at a sub-therapeutic amount, where the co-administration of the several compounds produces a reduction in blood pressure to a normal blood pressure or as close as possible to a normal blood pressure without causing symptoms of hypoperfusion. In further embodiments, the compositions of the invention comprising KSD179019 and/or a KSD179019 analog and/or derivative and at least one additional anti-hypertensive agent are administered at a synergistically effective therapeutic amount, where the co-administration of the amounts of the components produces a larger reduction in blood pressure than the sum of the blood pressure reductions achieved by the separate administration of the component amounts.

The compositions of the instant invention can be co-administered or used in combination with any anti-hypertensive agent used by an ordinarily skilled clinician including, but not limited to, a diuretic, beta-blocker, ACE inhibitor, angiotensin II receptor blocker, calcium channel blocker, alpha blocker, alpha-2 receptor antagonist, central agonist, peripheral adrenergic inhibitor, and vasodilator.

Diuretics useful in combination with the instant composition include, but are not limited to, bumetanide, chlorthalidone, chlorthiazide, ethacrynate, furosemide, hydrochlorothiazide, indapamide, methyclothiazide, metolazone, and torsemide.

Beta-blockers useful in combination with the instant composition include, but are not limited to, acebutolol, atenolol, bisoprolol, carvedilol, esmilol, labetalol, metoprolol tartrate, metoprolol succinate, nadolol, nebivolol, penbutolol, propranolol, and sotalol.

ACE inhibitors useful in combination with the instant composition include, but are not limited to, benazepril hydrochloride, captopril, enalapril maleate, fosinopril sodium, lisinipril, moexipril, perindopril, quinapril hydrochloride, ramipril, and trandolapril.

Angiotensin II receptor blockers useful in combination with the instant composition include, but are not limited to, azilsartan, candesartan, eprosartan mesylate, irbesarten, losartan potassium, olmesartan, telmisartan, and valsartan.

Calcium channel blockers useful in combination with the instant composition include, but are not limited to, amlopidine besylate, clevidipine, diltiazem hydrochloride, felodipin, isradipine, nicardipine, nifedipine, nimodipine, nisoldipine, and verapamil.

Alpha blockers useful in combination with the instant composition include, but are not limited to, doxazosin, mesylate, prazosin hydrochloride, and terazosin hydrochloride.

Alpha-2 receptor antagonists useful in combination with the instant composition include, but are not limited to, methyldopa. Central agonists useful in combination with the instant composition include, but are not limited to, clonidine hydrochloride and guanfacine hydrochloride.

Peripheral adrenergic inhibitors useful in combination with the instant composition include, but are not limited to, guanadrel, guanethidine monosulfate, and reserpine.

Vasodilators useful in combination with the instant composition include, but are not limited to, hydralazine and minoxidil.

It is within the skill of the artisan to determine the dosages of the respective agents when used in combination therapy with the instant compositions. For example, the instant composition can be administered in combination with one or more additional anti-hypertensive agent such that the blood pressure of the subject is lowered to a normal range of about 120/80 mmHg. In the alternative, the instant composition can be administered in combination with one or more additional anti-hypertensive agent such that the blood pressure of the subject is lowered to a range of 120-140 mmHg systolic and 80-90 mmHg diastolic pressure to avoid symptoms of hypoperfusion in the subject. The determination of the rate of blood pressure reduction, i.e., to what extent the blood pressure is reduced and over which period of time is within the skill of the ordinary clinician and is to be adjusted to the requirements of the individual subject. For example, the method of the invention may comprise treating a subject having an excessively high blood pressure by administering one or more fast-acting anti-hypertensive agents to lower the blood pressure over short period of time followed by administering a composition of the instant invention comprising KSD179019 and/or a KSD179019 analog and/or derivative and, optionally at least one additional anti-hypertensive agent to maintain a reduced blood pressure over an extended period of time.

Advantageously, the compositions of the instant invention provide a sustained activation of SCTR by administering KSD179019 and/or a KSD179019 analog and/or a KSD179019 derivative leading to, for example, a reduction in blood pressure such that excessive oscillations in blood pressure can be avoided. Therefore, the compositions of the invention are particularly suited for long-term blood pressure control.

Furthermore, compositions and methods are provided for the sustained activation of SCTR and sustained treatment of conditions or disease including, but not limited to, gastritis, acidity, gastrointestinal ulcers, pancreatitis and related disorders, liver cirrhosis, hepatoma, asthma, bronchitis, and diabetes by administering to a subject in need of such treatment the composition of the invention comprising KSD179019 and/or a KSD179019 analog and/or a KSD179019 derivative.

Further advantageously, the compositions of the instant invention comprising KDS179019 and/or a KDS179019 analog and/or a KSD179019 derivative are non-toxic and have no carcinogenic effects.

In preferred embodiments, methods are provided for identifying compounds that enhance secretin binding to the SCTR and prolong SCTR activation and include, but are not limited to, the design and manufacture of KSD179019 analogs and/or KSD179019 derivatives. Such KSD179019 analogs and KSD179019 derivatives are designed to enhance the specific interaction of the KSD179019 analog and derivative with the SCTR to reduce off-target effects and to increase the duration of association of secretin with the SCTR and/or reduce the dissociation of secretin from the SCTR.

In some embodiments, the methods of the invention comprise (a) using a computer program to generate a three-dimensional structure of the SCTR with a secretin peptide bound to the N-terminal region, (b) optionally, docking a cyclic cWDN tripeptide on to the secretin-bound SCTR model, (c) screening compounds, for example, derived from a small molecule compound library for a compound that interacts with the N-terminal and/or transmembrane region of the secretin-bound SCTR; and (d) testing the compounds of (c) which compounds interact with the N-terminal and/or transmembrane region of the secretin-bound SCTR by in vitro and in vivo assays for their ability to enhance and/or prolong secretin binding to the SCTR and/or reduce the dissociation of secretin from the SCTR.

Publicly available small molecule libraries used include, but not limited to, PubChem, CHEMBL, MolProt, ChemDiv, ChemSpace, NCI open and ZINC.

In other embodiments, methods are provided for using structural similarity testing for identifying small molecules that have structural similarity to KSD179019. Publicly available search engines for structural similarity include, but not limited to, ChemAxon PASS and Pubchem substructure searches.

In other embodiments, the methods of the invention comprise (a) using a computer program to generate a three-dimensional structure of a transmembrane region and N-terminal region of SCTR with a secretin peptide bound to the N-terminal domain of SCTR, (b) docking KSD179019 on the secretin-bound SCTR; (c) identifying molecular interactions between KSD179019 and specific amino acids of the secretin-bound SCTR; (d) introducing molecular modifications into KSD179019 at the sites of molecular interaction with the secretin-bound SCTR to generate KSD179019 analogs and/or KSD179019 derivatives; (e) testing the KSD179019 analogs and/or KSD179019 derivatives by in vitro and in vivo assays for their ability to enhance and/or prolong secretin binding to the SCTR and/or reduced the dissociation of secretin from the SCTR.

Advantageously, three-dimensional models of the secretin-bound SCTR have been generated and have been used in the methods of the instant invention to identify the docking site of KSD179019 on the secretin-bound SCTR. Without wanting to be bound by theory it is suggested that enhancing and/or prolonging the interaction of KSD179019 with the secretin-bound SCTR induces enhanced and/or prolonged secretin interaction with the SCTR and enhanced and/or prolonged blood pressure reduction.

In other specific embodiments, the methods of the invention comprise; (a) using a computer program to generate a three-dimensional structure of KSD179019 analogs and/or KSD179019 derivatives modeled into the secretin-bound SCTR; (b) identifying molecular interactions of portions of KSD179019 analogs and/or KSD179019 derivatives with the secretin-bound SCTR; (c) identifying modifications of the portions identified in (b) to enhance interactions and/or prolong interactions of the KSD179019 analogs and/or KSD179019 derivatives with the secretin-bound SCTR; d) introducing at least one identified molecular modification into the at least one portion of the KSD179019 analogs and/or KSD179019 derivatives to generate second-generation KSD179019 analogs and/or derivatives based on the modifications identified in (c); and (e) testing the second generation KSD179019 analogs and/or derivatives using in vitro and in vivo assays for testing the ability of the KSD179019 analogs and/or derivatives to enhance secretin binding to the SCTR and/or reduced secretin dissociation from SCTR.

The three-dimensional computer-generated models of the secretin-bound SCTR bound to KSD179019 analogs and/or KSD179019 derivatives are used to identify portions in the KSD179019 analogs and/or KSD179019 derivatives that are in close proximity to amino acids of the secretin-bound SCTR and/or are in positions on the KSD179019 analogs and/or derivatives, which positions lend themselves to molecular modifications that affect the KSD179019 analog and/or KSD179019 derivative interactions with the secretin-bound SCTR in a desired manner such that enhancement and/or prolongation of KSD179019 analog and/or derivative interaction with the secretin-bound SCTR is achieved. Advantageously, the three-dimensional computer-generated models can also be used to test the combination of more than one modification introduced into the KSD179019 analogs and/or derivatives to affect and/or enhance and/or prolong KSD179019 analog and/or KSD179019 derivative interactions with the secretin-bound SCTR.

Furthermore, the first and second generation KSD179019 analogs and/or KSD179019 derivatives can be used in three-dimensional computer-generated models of non-SCTR receptors to assess interactions or lack thereof of the KSD179019 analogs and/or KSD179019 derivatives with such non-SCTR receptors. In case of interactions, methods are provided to identify molecular interactions between the KSD179019 analogs and/or KSD179019 derivatives and such non-SCTR receptors and modify the KSD17919 analogs and/or KSD179019 derivatives to reduce or eliminate such undesired interaction of the KSD179019 analogs and/or KSD179019 derivatives with said non-SCTR receptors. Therefore, the methods provided enable optimization of KSD179019 analogs and/or KSD179019 derivatives for enhanced and/or prolonged interaction with secretin-bound SCTR and reduced or eliminated interactions with non-SCTR receptors.

In one embodiment, a method for making a KSD179019 analog and/or KSD179019 derivative is provided, the method comprising: providing a three-dimensional (3D) model of a human secretin-bound SCTR; virtually docking KSD179019 into the 3D model of the secretin-bound SCTR using binding energy estimation; selecting at least one portion of KSD179019 that, based on the virtual docking, is located in close proximity to at least one amino acid of KSD179019 docking site on the secretin-bound SCTR; introducing at least one molecular modification into the at least one selected KSD179019 portion to generate a KSD179019 analog and/or derivative; virtually docking the KSD179019 analog and/or derivative into the docking site using binding energy estimation; and chemically manufacturing the KSD179019 analog and/or derivative provided that the KSD179019 analog and/or derivative virtually docks into the docking site with a binding energy estimate that is lower than the binding energy estimate of KSD179019.

In some embodiments, methods are provided for identifying molecular modifications of the KSD179019 molecule to generate KSD179019 analogs and/or derivatives, which methods comprise modifying portions of the KSD179019 molecule that have been identified to be in close proximity to amino acids of the transmembrane region of the secretin-bound SCTR, wherein the modification(s) of the KSD179019 portions include, but are not limited to, atom and/or chemical group exchanges that add or remove a hydrogen donor or hydrogen acceptor, atom and/or chemical group exchanges that add or remove an aromatic group, and atom and/or chemical group exchanges that add or remove a hydrophobic group.

The modifications of the instant invention increase the interacting molecular forces between the KSD179019 molecule and the secretin-bound SCTR and/or reduce the dissociation of the KSD179019 molecule from the secretin-bound SCTR.

The methods of the instant invention further comprise manufacturing compounds designed according to the methods of the invention which compounds include, but are not limited to, KSD179019 analogs and/or KSD179019 derivatives with enhanced interaction with the secretin-bound SCTR, reduced dissociation from the secretin-bound SCTR, enhanced allosteric effect on secretin binding to the SCTR, and enhanced reduction in blood pressure.

Advantageously, an increase in affinity and/or reduction in dissociation of a KSD179019 analog and/or derivative improve allosteric site specificity, reduce off-site side effects and enable extended allosteric effects at lower doses.

Therefore, further provided are methods for manufacturing KSD179019 analogs and/or derivatives, which analogs and derivatives have similar properties and/or functions as KSD179019. In further embodiments, methods are provided for manufacturing KSD179019 analogs and/or derivatives, which analogs and/or derivatives have reduced undesired effects compared to KSD179019. In preferred embodiments, methods are provided for manufacturing KSD179019 analogs and/or derivatives, which analogs and/or derivatives have similar properties and/or functions as KSD179019 but reduced undesired effects compared to KSD179019.

### Definitions

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” “consisting essentially of,” “consists essentially of,” “consisting,” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims unless otherwise stated the term “about” meaning within an acceptable error range for the particular value should be assumed.

When ranges are used herein, such as for dose ranges, combinations and sub combinations of ranges (e.g., subranges within the disclosed range), specific embodiments therein are intended to be explicitly included.

“Treatment” or “treating” (and grammatical variants of these terms), as used herein, refer to an approach for obtaining beneficial or desired results including but not limited to therapeutic benefits. A therapeutic benefit is achieved with the eradication or amelioration of one or more of the physiological symptoms associated with hypertension such that an improvement is observed in the subject, notwithstanding that, e.g., a subject suffering from secondary hypertension may still be afflicted with the underlying disease or condition.

As used herein, “therapeutic agent” refers to KSD179019 or a KSD179019 analog or derivative and pharmaceutically acceptable carrier, optionally, in combination with one or more anti-hypertensive agents.

A “therapeutic effect,” as used herein, encompasses a therapeutic benefit as described above. This includes delaying the appearance of elevated blood pressure or hypertension, delaying the onset of symptoms associated with an elevated blood pressure or hypertension, slowing, halting, or reversing the progression of an elevated blood pressure or hypertension, or any combination thereof.

A “therapeutically effective amount” of a composition is the amount that results in a therapeutic effect in the subject and can be determined by monitoring the blood pressure of the subject and by monitoring the existence, nature, and extent of any adverse side effects that accompany the administration of the composition; the LD₅₀ of the composition; and the side effects of the composition at various concentrations.

A “synergistically effective” therapeutic amount or “synergistically effective” amount of KSD179019 or a KSD179019 analog or derivative and pharmaceutically acceptable carrier is an amount which, when combined with an effective or sub-therapeutic amount of one or more anti-hypertensive agents, produces a greater blood pressure reduction than the sum of blood pressure reducing effects achieved when either the KSD179019 or a KSD179019 analog or derivative or the one or more anti-hypertensive agent are used alone. Therefore, a synergistically effective therapeutic amount of KSD179019 or a KSD179019 analog or derivative and one or more anti-hypertensive agents produces a greater effect when used in combination than the additive effects of each of the KSD179019 or a KSD179019 analog or derivative or the one or more agents when used alone. The term “greater effect” encompasses not only a reduction in symptoms, e.g., a reduction in hypertension, but also reduced side effects, improved tolerability, improved subject compliance, improved efficacy, or any other improved clinical outcome.

A “sub-therapeutic amount” of KSD179019 or a KSD179019 analog or derivative and a pharmaceutically acceptable carrier is an amount less than the effective amount, but which when combined with an effective or sub-therapeutic amount of the one or more additional anti-hypertensive agents can produce a desired result, due to, for example, synergy in the resulting efficacious effects (e.g., therapeutic benefit) for the subject, or reduced side effects associated with the compounds administered to the subject. Typical therapeutic amounts for KSD179019 or a KSD179019 analog or derivative and pharmaceutically acceptable carrier, optionally, in combination with one or more anti-hypertensive agent can be ascertained from various publicly available sources and/or routine experimentation.

The terms “co-administration,” “administered in combination with,” and their grammatical equivalents encompass administration of two or more agents to a subject so that both agents and/or their analogs or derivatives are present in the subject at the same time. Co-administration includes simultaneous administration in separate compositions, administration at different times in separate compositions, or administration in a composition in which more than one agent is present. Co-administered agents may be in the same formulation. Co-administered agents may also be in different formulations.

The terms “simultaneous” or “simultaneously” as applied to administering agents to a subject refer to administering one or more agents at the same time, or at two different time points that are separated by no more than 1 hour. The term “sequentially” refers to administering more than one agent at two different time points that are separated by more than 1 minute, e.g., from about 2 minutes to about 60 minutes; about 5 minutes to about 50 minutes; about 8 minutes to about 40 minutes; about 10 minutes to about 30 minutes or about 15 minutes to about 20 minutes; or from about 1 hour to about 12 hours; about 2 hours to about 10 hours; about 3 hours to about 8 hours; about 4 hours to about 7 hours; or about 5 hours to about 6 hours or even longer.

The KSD179019 or a KSD179019 analog or derivative can also be administered in the presence of a salt. When compounds of the present invention contain relatively acidic functionalities, base addition salts can be obtained by contacting the neutral form of such compounds with a sufficient amount of the desired base. Examples of pharmaceutically acceptable base addition salts include, but are not limited to, sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid. Examples of pharmaceutically acceptable acid addition salts include, but are not limited to, those derived from inorganic acids like hydrochloric acid, hydrobromic acid, perchloric acid, nitric acid, thiocyanic acid, carbonic acid, monohydrogencarbonic acid, phosphoric acid, monohydrogenphosphoric acid, dihydrogenphosphoric acid, sulfuric acid, monohydrogensulfuric acid, hydriodic acid, or phosphorous acid and the like, as well as the salts derived from relatively nontoxic organic acids like acetic acid, propionic acid, isobutyric acid, maleic acid, malonic acid, benzoic acid, succinic acid, suberic acid, fumaric acid, mandelic acid, phthalic acid, benzenesulfonic acid, p-tolylsulfonic acid, citric acid, tartaric acid, methanesulfonic acid, and the like. Also included are salts of amino acids such as arginate and the like, and salts of organic acids like glucuronic acid or galactunoric acid and the like.

Certain specific compounds of the present invention contain both basic and acidic functionalities that allow the compounds to be converted into either base or acid addition salts. The neutral forms of the compounds may be regenerated by contacting the salt with a base or acid and isolating the parent compound in a conventional manner. The parent form of the compound differs from the various salt forms in certain physical properties, such as solubility in polar solvents, but otherwise, the salts are equivalent to the parent form of the compound for the purposes of the present invention.

“Pharmaceutically acceptable carrier” or “pharmaceutically acceptable excipient” includes any and all solvents, dispersion media, coatings, antibacterial and antifungal agents, isotonic, and absorption delaying agents, and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

The amount of KSD179019 or a KSD179019 analog or derivative administered can be an amount from a low of about 0.01 mg/day, about 5 mg/d or about 10 mg/d to a high of about 750 mg/d, about 800 mg/d or about 1 g/d. For example the amount of KSD179019 or a KSD179019 analog or derivative can be from about 0.01 mg/d to about 1 g/d, about 10 mg/d to about 800 mg/d, about 20 mg/d to about 500 mg/d, about 30 mg/d to about 400 mg/d, about 40 mg/d to about 300 mg/d, about 50 mg/d to about 200 and/d, about 60 mg/d to about 100 mg/d; or about 0.01 mg/d to about 10 mg/d, about 0.02 mg/d to about 20 mg/d, 0.03 mg/d to about 30 mg/d, 0.04 mg/d to about 40 mg/d, about 0.05 mg/d to about 50 mg/d, about 0.06 mg/d to about 60 mg/d, about 0.07 mg/d to about 70 mg/d, about 0.08 mg/d to about 80 mg/d, about 0.09 mg/d to about 90 mg/d, and about 0.1 mg/d to about 100 mg/d.

The amount of KSD179019 or a KSD179019 analog or derivative administered in a composition with one or more additional anti-hypertensive agents can be any of the amounts used when KSD179019 or a KSD179019 analog or derivative is administered alone or can be reduced proportionally to the increase of the one or more additional anti-hypertensive agents co-administered. The methods of the invention include measuring the blood pressure during and continuously or discontinuously after administering a composition of the instant invention to monitor the effect of the composition and, in case of rapid or extensive reduction in blood pressure below a value of 100/50 mmHg to administer an agent that either block further reduction of blood pressure or increases blood pressure. Such agents are within the knowledge of the skilled clinician.

“Subject” refers to an animal, such as a mammal, for example, a human. The methods described herein can be useful in both pre-clinical and clinical human therapeutics and veterinary applications. In some embodiments, the subject is a mammal (such as an animal model of disease), and in some embodiments, the subject is human. Non-limiting examples of subjects include canine, porcine, rodent, feline, bovine, poultry, equine, human, and a non-human primate.

The instant invention also provides compounds that are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the instant invention when placed in a transdermal patch reservoir with a suitable enzyme or chemical reagent.

In some embodiments, the therapeutically effective amount of said pharmaceutical composition can be administered through oral, rectal, bronchial, nasal, topical, buccal, sublingual, transdermal, vaginal, intramuscular, intraperitoneal, intravenous, intra-arterial, intracerebral, intraocular administration or in a form suitable for administration by inhalation or insufflation, including powders and liquid aerosol administration, or by sustained release systems such as semipermeable matrices of solid hydrophobic polymers containing the compound(s) of the invention. Administration may be also by way of other carriers or vehicles such as patches, micelles, liposomes, vesicles, implants (e.g. microimplants), synthetic polymers, microspheres, nanoparticles, and the like.

In some embodiments, the compositions of the instant invention may be formulated for parenteral administration e.g., by injection, for example, bolus injection or continuous infusion. In addition, the composition may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g. sterile, pyrogen-free water, before use.

In some embodiments, the compositions of the instant invention may be formulated in aqueous solutions for oral administration. The composition may be dissolved in suitable solutions with added suitable colorants, flavors, stabilizing and thickening agents, artificial and natural sweeteners, and the like. In addition, the composition may further be dissolved in solution containing viscous material, such as natural or synthetic gums, resins, methylcellulose, sodium carboxymethylcellulose, or other well-known suspending agents.

In other embodiments, the compositions of the instant invention are applied topically or systemically or via a combination of both. The compositions may be formulated in the forms of lotion, cream, gel and the like.

In some embodiments, the compositions of the instant invention can be applied directly to the nasal cavity by conventional means, for example with a dropper, pipette or spray. The compositions may be provided in the single or multi-dose form. Administration to the respiratory tract may also be achieved by means of an aerosol formulation in which the active ingredient is provided in a pressurized pack with a suitable propellant such as a chlorofluorocarbon (CFC), e.g., dichlorodifluoromethane, trichlorofluoromethane, or dichlorotetrafluoroethane, carbon dioxide, or other suitable gas. The aerosol may conveniently also contain a surfactant such as a lecithin. Furthermore, the compositions may be provided in the form of a dry powder, e.g., a powder mix of the compound in a suitable powder base such as lactose, starch, starch derivatives such as hydroxypropyl methyl cellulose and polyvinylpyrrolidone (PVP). Conveniently, the powder carrier will form a gel in the nasal cavity. The powder composition may be presented in unit dose form, e.g., in capsules or cartridges of gelatin, or blister packs from which the powder may be administered by means of an inhaler.

In some embodiments, the pharmaceutical compositions are provided in unit dosage forms, wherein the composition in the desired form is divided into unit doses containing appropriate quantities of the active component. The unit dosage form can be a packaged preparation, the package containing discrete quantities such as packaged tablets, capsules, and powders in vials or ampoules. Moreover, the unit dosage form can be a capsule, tablet, cachet, or lozenge itself, or it can be the appropriate number of any of these in packaged form. In preferred embodiments, tablet or capsule forms are for oral administration and liquid form are for intravenous administration and continuous infusion.

The instant invention also provides kits comprising the compounds and/or pharmaceutical compositions as described herein. The kits may further be used in the methods described herein. The kits may also include at least one reagent and/or instruction for their use. The kits may include one or more containers filled with one or more compounds and/or pharmaceutical composition described in the present invention and may also comprise a control composition, such as a known anti-hypertensive agent.

Pharmaceutical compositions for topical administration of a composition of the instant invention, optionally, in combination with one or more additional anti-hypertensive agents to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, e.g., include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels may include an aqueous carrier base and a gelling agent such as a cross-linked polyacrylic acid polymer, a derivatized polysaccharide (e.g., carboxymethyl cellulose), and the like.

Pharmaceutical compositions suitable for topical administration in the mouth (e.g., buccal or sublingual administration) comprise the compositions of the instant invention in a flavored base, such as sucrose, acacia, or tragacanth; pastilles comprising the composition in an inert base such as gelatin and glycerin or sucrose and acacia; and mouthwashes comprising the active ingredient in a suitable liquid carrier. The pharmaceutical compositions for topical administration in the mouth can include penetration enhancing agents if desired.

### Materials and Methods

### Pharmacophore Modeling at SCTR:

We developed the 3D structure of SCTR in complex with secretin peptide by the help of homology modeling and virtual docking [41]. The receptor structure docked with secretin peptide was visualized by Schrodinger maestro suite. The active site pre-defined from SCTR-secretin model was analyzed to locate the amino acid residues interacting to secretin peptide. After that, the most important residues were defined and used to develop the pharmacophore model by the help of Pharmit [42]. The cyclic tripeptide (cWDN), with known agonistic activity for SCTR [43], was docked on to SCTR model via patchdock/firedock algorithm. This complex was used as the principal structure for the screening of small compound with agonistic potential.

### In Silico Screening of Potential Modulators:

The SCTR pharmacophore model was then used for in silico screening of small compounds in the Pharmit database. The screening was performed on all available confirmations of 14,392,554 compounds present in CHEMBL, Chem-Div, MolProt, NCI open, Chem-Space, NCI open, PubChem and Zinc databases. The selected compounds were used in Pubchem structure search for similar compounds.

The screening processes are summarized here. The docking was performed using two different algorithms for binding energy calculations. The first docking algorithm used was iGemDock, generally used to screen large library of compounds against a pre-defined active site [44]. The second docking algorithm used was Autodock Vina. It is the most commonly used algorithm for the screening of large compound library [45]. PyRx dock was used as an interface to run Autodock on windows platform [46]. The PyRx dock was used by keeping all the other parameters at default. Scoring of the docked compound was done using their individual binding score calculated from docking algorithms. The structures with more than 100 binding score as calculated by iGemDock were selected. Final screening was performed with binding affinity calculated by AutoDock Vina. The compounds with more than −8.0 binding affinity were selected. The selected compounds after the docking algorithms were combined and further screened for their binding location on the full receptor. Further validation of the binding pocket was done by the help of patchdock/firedock algorithm [47].

### In Vitro Functional Assay of KSD179019:

As SCTR is known to have cAMP as the secondary messenger, therefore to study receptor activation, the concentration of cAMP is measured by the help of Lance cAMP assay using the assay kit from PerkinElmer, as per the standardized protocol in our lab [41, 48]. The observed results were reported as the fold change in the intracellular cAMP levels when compared to the basal level (no agonist control). Additionally, the cells upon transfection were used for the extraction of total RNA (TriPure reagent, Invitrogen) to confirm the receptor's expression.

### Ex-Vivo Functional Analysis of KSD179019:

Tissues which are known to express high levels of SCTR, such as duodenum, pancreas, and cerebellum were used to study the effect of KSD179019. Various tissues were collected from C57 mice and were washed three times with ice-cold HBSS (Invitrogen). Tissues were minced and weighed equally into four parts. These parts were treated with 100 μM KSD179019, secretin (1 μM), 100 μM KSD179019 and secretin (1 μM), and buffer alone as basal cAMP levels. After drug treatment, the tissues were homogenized for measuring the concentration of the cAMP by the Lance cAMP assay kit.

### In Vivo Functional Analysis of KSD179019:

The effect of intracerebroventricular (i.c.v) injection of KSD179019 was performed on the mice with i.c.v. cannula implant. The complete protocol was performed as reported in our lab [49, 50]. Mice were implanted with the stainless-steel cannula with a projection to the lateral ventricular region (bregma: 0.5 mm, lateral: 1.0 mm and depth: 2.0 mm). The mice were allowed to recover for at least 5 days after the surgery. Artificial cerebrospinal fluid (ACSF) prepared according to Alzet protocol, was used as a vehicle for the administration of the KSD179019 (260 ng/5 μl) and secretin (500 ng/5 μl) (AnaSpec, Fremont, Calif., USA). ACSF alone was used as a vehicle control. After drug treatment, blood was collected from the jugular vein at 0, 15, 30, 45 and 60 min. Plasma samples were collected from the whole blood by centrifugation at 4000×g for 10 min at 4° C. The samples were then used for the quantification of vasopressin by the help of ELISA using a commercial kit from (Phoenix Pharmaceuticals, Inc.) according to manufacturer instruction.

### Anti-Hypertensive Effect of Secretin:

The in vivo effects of secretin peptide (0.5 μg/g BW) on the blood pressure of C57 mice were monitored in real-time and continuously by the help of DSI telemetry transplant (datasci.com). The DSI telemetric device was implanted using the standardized protocol from our lab [51]. Post recovery, blood pressure measurements, and drug administration were initiated. Secretin was administered intra-peritoneal at the concentration of 0.5 μg/g BW on the animal preinstalled with the DSI device. The heart rate and blood pressure were measured continually up to 150 min post-injection time.

### Anti-Hypertensive Action of KSD179019:

Anti-hypertensive action of KSD179019 was analyzed by continues monitoring of the blood pressure and heart rate on spontaneously hypertensive rat (SHR) by using DSI telemetric implantation. DSI device was surgically implanted using the methodology described before. KSD179019 was administered intravenously at dose of 0.26 μg/g BW.

Comparative Analysis of KSD179019 with Conventional Drugs:

We used conventional combination therapy i.e. Azilsartan (Az; 0.3 μg/g BW) and Chlorthalidone (CLT; 1 μg/g BW) for the comparison with KSD171990. Az is an angiotensin II receptor antagonist and CLT is a diuretic compound. Both these compounds are commonly used in the treatment of hypertension either individually and in combination [52]. The dosing of the combination of Az+CLT on SHR rats is used as documented in previous publications [53]. For blood pressure monitoring, telemetric device by DSI was used, using the implantation methodology mentioned earlier [51].

Toxicity and Carcinogenicity Studies

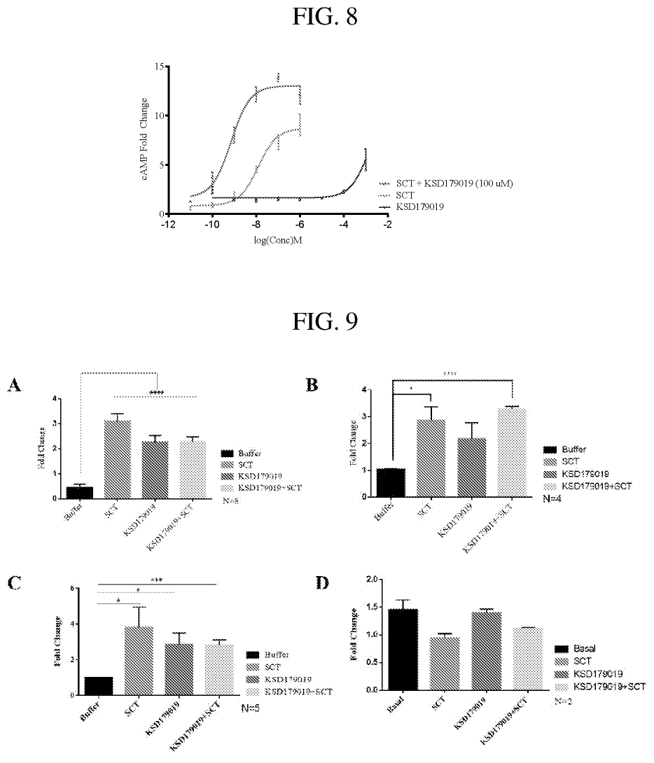

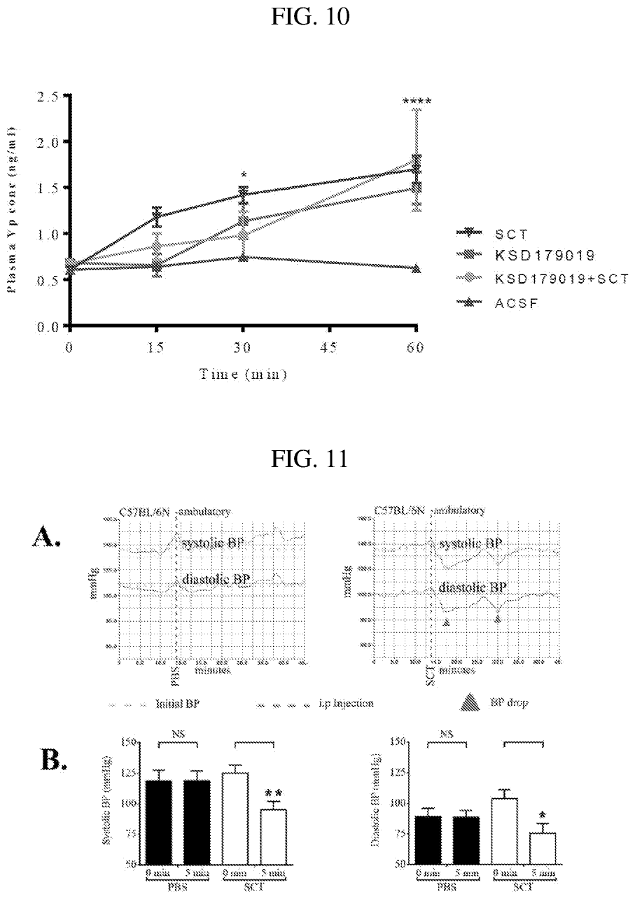

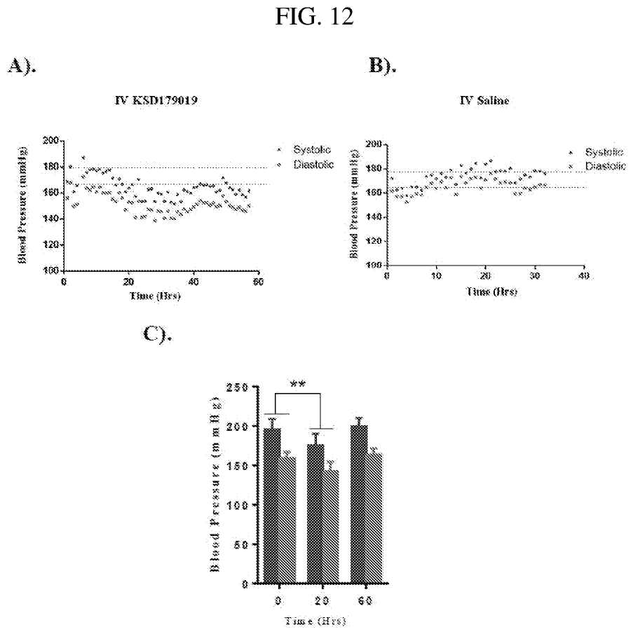

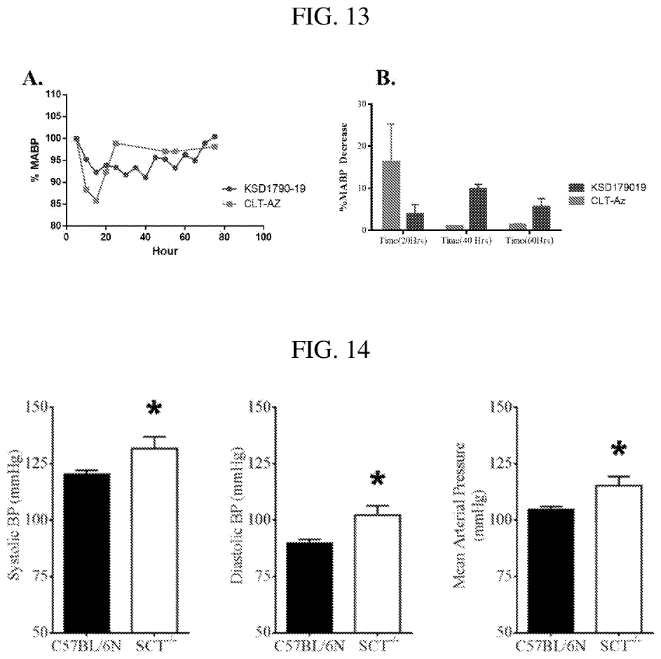

In vitro and in vivo toxicity testing was performed. All these test were performed using the OECD guidelines. For in vitro testing, KSD179019 organ/cell specific toxicity was determined using a cell viability testing (i.e. MTT assay). The MTT assay was performed on liver cells (HepG2 cell line) and kidney cells (HEK293 cell line) as they are the major organs effected by drug toxicity. It was observed that KSD179019 has no toxic effect on these cells at dose ranging from 100 nM to 1 mM (FIG. 7a; 7b). Additionally, the potential carcinogenicity of this drug is tested according to OECD-471 (Ames test) using the commercially available kit from XENOMETRIX. The mutation effect of the drug was tested in salmonella strains (TA98, TA100, TA1535 and TA1537) and on the combination of two E.coli strains (uvrA and pKM101) (FIGS. 8a; 8b; 8c; 8d and 8e). No mutational effect of KSD171990 was observed after the testing. Additionally, as the drug's secondary metabolite can also have carcinogenic effect, the Ames test was performed in presence of liver enzymes which can cause the formation of secondary metabolites (S9 liver extract) (FIGS. 9a; 9b; 9c; 9d and 9e). The results clearly show that there is no carcinogenic potential of KSD179019 or its metabolites. Further, in vivo toxicological test of KSD179019 were performed. The animal testing was done under an animal license from the Laboratory Animal Unit (LAU) of the University of Hong Kong for testing at acute, sub-chronic and chronic levels of exposure. The study was done based on the OECD-423 guidelines. The acute toxicity of KSD179019 was performed with single oral administration 2000 mg/kg BW (i.e. highest dose recommended by OECD to be tested for LD50 estimation) drug to estimate the Lethal Dose 50 (LD50) for the drug. After the KSD179019 administration, the animals were observed for food intake, water intake, digestion/constipation, urination, body temperature, change in skin and fur, drowsiness, aggressiveness, sleep, rate of respiration/breathing, eye color, ears, diarrhea, heartbeat, activity, general physique, coma, injury, hematochezia (blood stool), loss of appetite, convulsions, salivation, body weight, general reflex (CNS/ANS) and death (FIG. 10). In the course of the investigative period of 14 day after single dose of 2000 mg/kg BW, KSD179019 showed no effect on the overall health of animals. The test was done on 6 animals in two batches in both males and females. The body weight change remained under 2 grams (FIG. 11) which is normal growth for 9 weeks old C57BL/6J (https://www.jax.org/jax-mice-and-services/strain-data-sheet-pages/body-weight-chart-000664#) [66]. The body temperature (FIG. 12) remained constant for all the animals under observation. Furthermore, since no animal fatality was observed, the LD50 was estimated to be between 2000-5000 mg/kg BW. The animals were subjected to histological analysis post sacrificing on day 14. Major organs like brain, heart, liver, kidney, intestine, and colon were collected and analysed. None of these organs were observed to have any signs of necrosis. This observation indicated that the compound KSD179019 was well tolerated in the rodents. However, in order to determine a dose for sub-chronic and chronic studies, according to OECD recommendations, 3 animals were administered 5000 mg/kg BW of drug in a single administration. The test was performed on both male and female C57BL/6J mice and no fatal or toxic effects were observed. During the 14 day observation the animals remained healthy with no change in body weight and body temperature (FIG. 13).

### EXAMPLES

### Example 1: Uses of Compounds of the Instant Invention

### Hypertension and Improved Cardiac Profile:

Secretin is known to reduce blood pressure and improve the cardiac profile [12, 13]. Moreover, reports of synthetic secretin triggering a reduction of blood pressure as one of its side effects during clinical trials is documented [14]. Its interaction with SCTR involves nitric oxide pathway and leads to enhancement in cardiac perfusion. This phenomena reported in pigs provides a positive correlation between NO production and cAMP/PKA signaling [15]. Secretin treatment was found to be useful for enhancing the cardiac output in left ventricular failure patients [16]. Clinical trials with secretin has shown that its infusion increases stroke volume, renal blood flow and reduces systemic resistance and hence, it may be concluded that secretin has a great potential for cardiac therapeutic research [16]. Diuretics are known to be as the most common medication treatment for hypertension patients and secretin peptide is also found to act as a diuretic in human trials [17]. Secretin is known to mediate water homeostasis at three different levels, i.e. hypothalamus, pituitary and kidney [18]. This physiological effect of secretin was confirmed by the presence and expression of secretin along with its receptor and its co-localization with vasopressin in the hypothalamic PVN and SON [5]. The role of secretin as a diuretic was debated [19], but confirmed by the help of secretin knockout mice (SCT−/−) and secretin receptor knockout mice (SCTR−/−) in our laboratory [18, 20]. Therefore, the potential of secretin peptide as an anti-hypertensive molecule was explored.

### SCTR Modulator:

The use of secretin peptide directly as a therapeutic compound is limited because of the short half-life (approximately 2 min) in circulation. This limitation has partially been overcome by the development of synthetic secretin peptide analogs SecreFlo™ and ChiRhoStim®, with a biological half-life of 20 min and 45 min, respectively. The short half-life of secretin and analogs in circulation led the inventors to search for small compound agonists with extended half-life, and the inventors identified a positively allosteric modulator (PAM), KSD179019, of the receptor. PAMs are capable of positively modulating the activation of a receptor in the presence of agonist molecule by binding at an allosteric site, and hence, allosteric modulators are known to possess relatively low side effects because of their unique binding sites [21]. Apart from hypertension, secretin and SCTR are involved in several other pathological conditions. Therefore, KSD179019 has therapeutic potential for other clinical conditions as well. Below, the roles of secretin in different pathophysiological conditions are briefly described.

### Gastritis, Acidity and Gastrointestinal Ulcers:

Secretin is an enterogastrone to inhibit acid release from parietal cells in the stomach [22, 23]. KSD179019, therefore, has the potential to develop as an anti-ulcer drug.

### Pancreatitis and Related Disorders:

Secretin induces both positive and negative feedback regulation on pancreas [24], and it has been proposed as a therapeutic molecule against acute pancreatitis and has reached its way to the clinical trials [25-27]. KSD179019 can be developed for the treatment of acute pancreatitis.

### Liver Cirrhosis and Hepatoma:

The secretion of bile, water, and salts from the liver is stimulated by secretin along with the cAMP response in isolated cholangiocytes [28]. Also, the binding of secretin to its receptor in liver cholangiocytes is well documented [29, 30]. Clinical studies have proved secretin to be of therapeutic value in intrahepatic cholestasis [31] and also secretin-induced bile secretions can stimulate liver cell regeneration after hepatectomy [32].

### Asthma and Bronchitis:

SCTR levels are found to elevate in asthmatic conditions. Additionally, secretin hormone is reported to be effective in the treatment of asthmatic brachiates via activation of anionic efflux (Google Patents, US20040241154A1). In humans, secretin stimulates the movement of chloride anions in epithelial cells and therefore, secretin is wildly tested as a therapeutic agent in asthma at the preclinical stage [33]. The preclinical trial in Pharmagen drug discovery research suggests that secretin stimulates the movement in epithelial bronchus and causes broncho relaxation [34].

### Diabetes:

Secretin is known to elevate the level of insulin and downregulate the levels of glucagon hormone [35] and was validated in human studies where the elevated glucose levels were effectively reduced by secretin [36-38]. The effectiveness of secretin in elevating the insulin levels is reported in preclinical and clinical studies [36, 39]. However, some contradictory reports in clinical trials on the effectiveness of secretin in alleviating the symptoms of diabetes were stated [40]. Therefore, further studies are required to clarify its potential in diabetic treatment.

Additionally, several patents have previously been granted on the therapeutic capability of secretin peptide:
Patent No./Title: Secretin receptor agonists to treat diseases or disorders of energy homeostasis (WO2017202851A1)
Publication Date: 30 Nov. 2017
Patent No./Title: Use of secretin-receptor ligands in treatment of cystic fibrosis (CF) and chronic obstructive pulmonary disease (COPD) (CN1923187A)
Publication Date: 7 Mar. 2007
Patent No./Title: Secretin for the treatment of asthma (WO2003011327A2)
Publication Date: 13 Feb. 2003
Patent No./Title: Use of secretin and secretin analogs to treat affective disorders. (WO2004081195A2)
Publication Date: 23 Sep. 2004

### Example 2: Pharmacophore Modeling

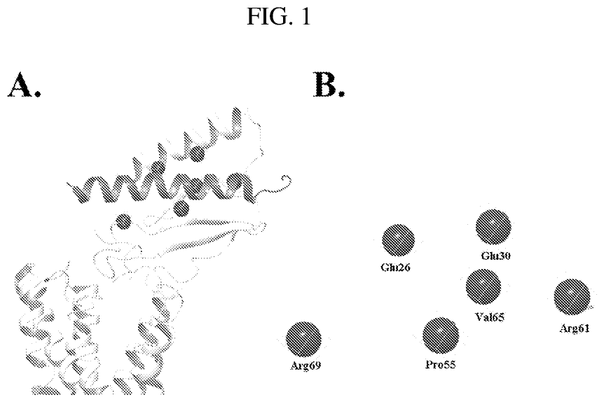

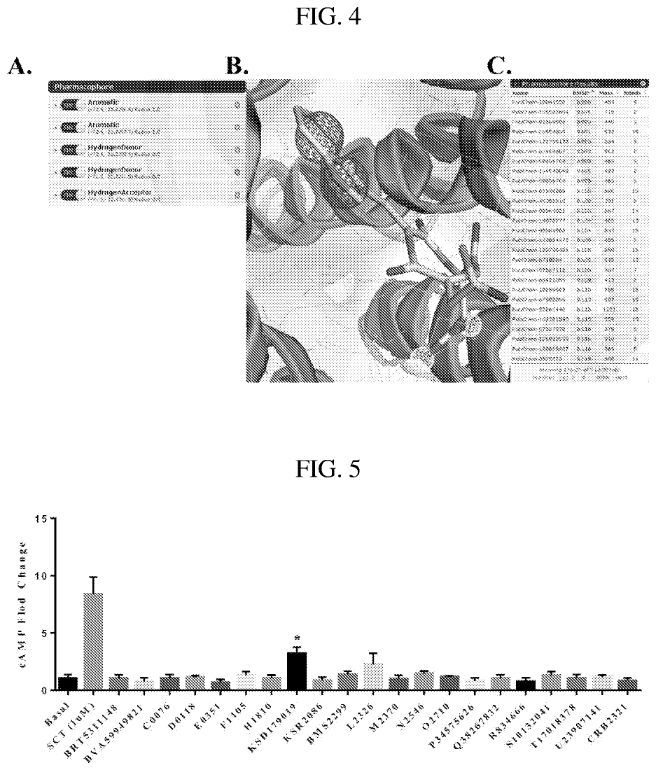

Secretin-SCTR binding interaction was studied by the help of available photoaffinity data and by the homology modeling of SCTR with secretin peptide docked on to it. This part of the work has already been published by our group [41]. It was shown that the full peptide interacts with the N-terminal extracellular domain of the SCTR, via residues Arg72, Gln22, Glu18, Arg69, Gln19, Alu26, Pre55, Gln30, Ser56, Leu35, Arg61, Met62, Val65, Glu66 and Glu64. The docked peptide-receptor model was used for the development of pharmacophore model using six crucial residues with highest binding potential (i.e. Glu26, Glu30, Pro55, Arg61, Val65 and Arg69) (FIG. 1). The details of selected pharmacophores are as follows: Glu26 as hydrogen donor and acceptor, Glu30 as hydrogen donor and hydrogen acceptor, Pro55 as aromatic, Arg61 as hydrogen donor, Val65 as hydrophobic pharmacophore and Agr69 as hydrogen donor. The second pharmacophore model created on the previously identified allosteric site by the help of cWDN peptide with weak intrinsic SCTR activation potential [43] (FIG. 3). The Pharmacophores were designed using the aromatic group of tryptophan and the carboxylic acid group attached to the side chain of the aspartic acid (FIG. 4A). Five pharmacophores were used in total for the development of the model. Two aromatic groups were used at the pentose and benzene ring of the tryptophan (FIG. 4B). The other three pharmacophores were identified on the side chain of aspartic acids. The oxygen atom was identified as hydrogen bond acceptor and alcohol group was identified as hydrogen bond donor and acceptor. This pharmacophore model was used to screen 6 major small molecule libraries using Pharmit tool [42].

### Example 3: Virtual Compound Library Generation

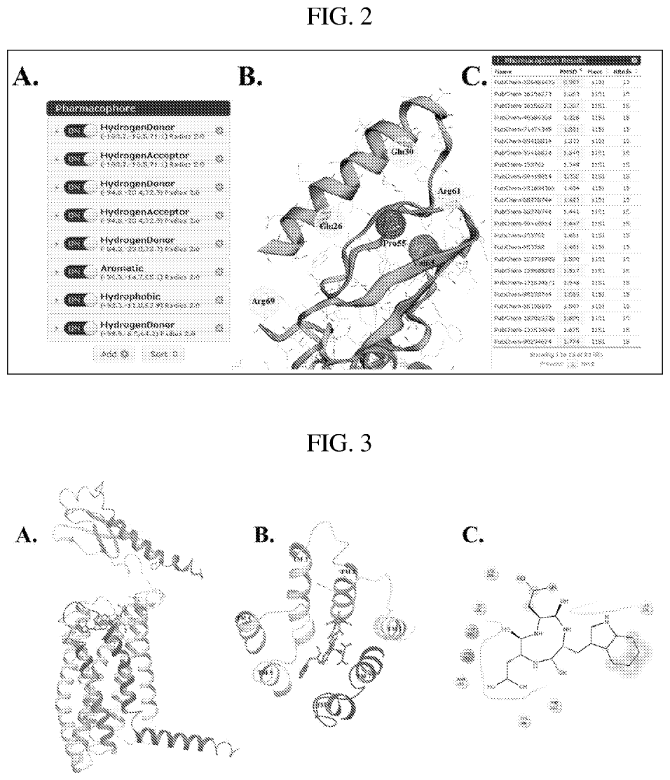

The screening was done from six small compound libraries with 14,392,554 compounds. For the secretin binding site, using the pharmacophore model we were able to obtain a total of 27 hits (23 from PubChem, 3 from CHEMBL and 1 from MolProt respectively) (FIG. 2). cWDN-based pharmacophore was used to screen for 1,03,528 hits (FIG. 4C). Hits from both the pharmacophore searches were combined together and a total of 1,03,555 small compounds were selected as hits (CHEMBL-11010hits; ChemDiv-330 hits; ChemSpace-8173, Molprot-5,163 hits; NCI open-422; PubChem-71,630; ZINC-6,827) via Pharmit tools. Apart from these we have also selected 870 compounds with structural similarity of cWDN compound by the help of ChemAxonPASS and from Pubchem substructure search. On the basis of the source, all the compounds were assigned a code and numbering for further screening.

### Example 4: Virtual Screening for SCTR Agonist

The library generated was docked by the help of two different virtual docking algorithms for the initial selection with the binding site predefined. The docking was performed first by iGemDOCK. The compounds with more the 100 binding affinity score were selected for screening from AutoDock Vina. Compounds selected from Vina were with higher than −8 ΔG value. Finally, 810 compounds were selected with the highest binding affinity. The patchdock/firedock algorithm (no predefined active site) were used to verify the binding site of the molecules on the SCTR structure. After all the in silico analysis, we have selected 21 compounds on the basis of binding affinity and binding location by the help of three independent docking algorithms (Table 1).

TABLE 1
List of the selected compound after in silico screening
of pharmacophore model based hits. 22 compounds
selected for in vitro analysis as SCTR modulators with
their assigned compound ID and their respective docking
score calculated from iGemDOCK and AutoDock respectively.

| | | Docking Score | |
|---|---|---|---|
| | Compound ID | iGemDOCK | AutoDock |
| 1 | BRT5311148 | 1298.64 | −9.7 |
| 2 | BVA59949821 | 1587.08 | −9.5 |
| 3 | C0076 | 519.12 | −9.8 |
| 4 | D0118 | 542.19 | −10.1 |
| 5 | E0351 | 553.61 | −9.8 |
| 6 | F1105 | 307.76 | −9.7 |
| 7 | H1810 | 716.22 | −10.1 |
| 8 | KSD179019 | 899.26 | −10 |
| 9 | KSR2086 | 540.80 | −10.1 |
| 10 | BMS2299 | 349.51 | −10 |
| 11 | L2326 | 590.12 | −10.4 |
| 12 | SAR2370 | 316.09 | −10.1 |
| 13 | N2546 | 521.19 | −10.4 |
| 14 | O2710 | 616.39 | −9.8 |
| 15 | P34575626 | 810.03 | −9.5 |
| 16 | Q38267832 | 696.75 | −10.4 |
| 17 | R834666 | 415.30 | −9.5 |
| 18 | S10132041 | 434.58 | −9.7 |
| 19 | T17018378 | 434.06 | −9.6 |
| 20 | U23907141 | 629.49 | −9.7 |
| 21 | CARB2312 | 569.06 | −9.6 |

### In Vitro Screening of Selected Compounds:

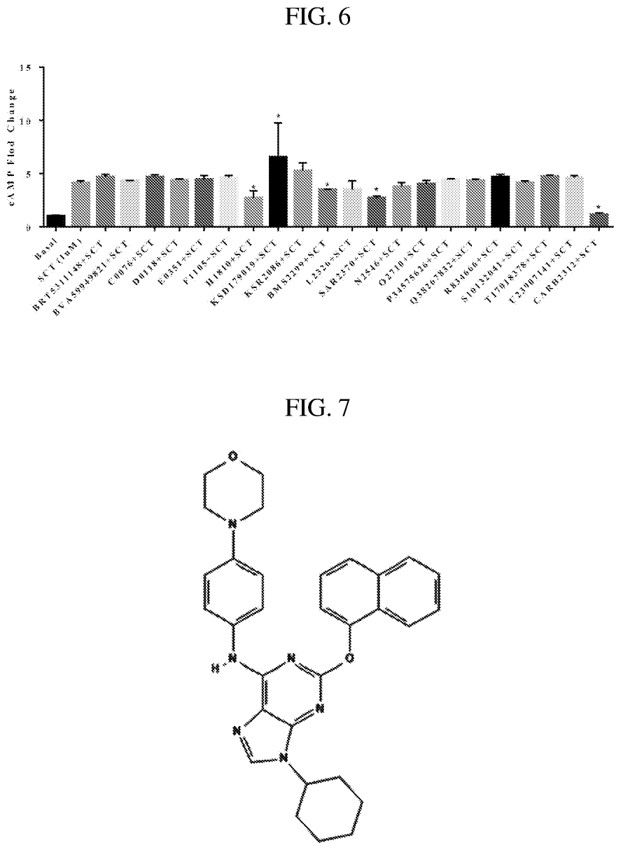

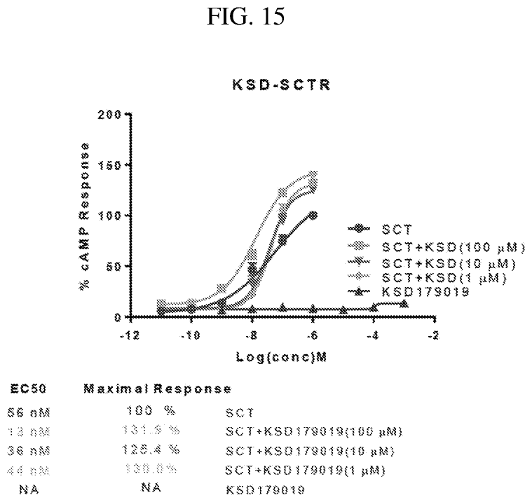

The selected compounds were procured from TargetMol and chemicalbook.com. For the screening of agonistic effect, the selected compounds were used at 100 μM concentration with secretin used at 1 μM concentration. We observed a low but statistically significant increase in cAMP concentration with one compound, KSD179019 (structure in FIG. 7) when compared to the basal level of cAMP concentration (FIG. 5). To find the antagonist, we added all the compounds at 100 μM along with 1 μM secretin peptide. KSD179019 potentiated the effect of secretin with significantly higher cAMP response (FIG. 6). After the initial in vitro screening, five different compounds were selected as potential SCTR modulator from the cAMP based receptor activation assay. The five compounds were then used for analyzing their dose-dependent response on the SCTR to verify their effect on the receptor. One of them, KSD179019, was capable of activating the SCTR in dose-dependent manner. KSD179019 showed a dose-dependent potentiation of secretin on cAMP stimulation (FIG. 8 and FIG. 15). It was therefore identified to be a positive allosteric modulator (PAM) and was selected for further ex vivo tests.

### Example 5: Ex Vivo Analysis of KSD179019

We analyzed the effect of KSD179019 on tissues with SCTR expression including duodenum, pancreases, cerebellum, and kidney. Secretin (1 μM) and KSD179019 (100 μM) were able to stimulate cAMP production from duodenum, cerebellum, and pancreas but not kidney (FIG. 9). In kidney, a synergistic effect of secretin and KSD179019 might not have been observed possibly because of receptor saturation due to low expression levels of the intrinsic receptor.

### Example 6: In Vivo Effect of KSD179019

To test the biological effect of KSD179019 comparing to secretin, the effect of i.c.v-KSD179019 on stimulating circulatory vasopressin (Vp) level was investigated [5]. Plasma Vp concentration versus time after i.c.v. secretin (500 ng/5 μl), KSD179019 (260 ng/5 μl) and secretin/KSD179019 administration (same doses), along with the vehicle control (ACSF) in C57 mice were measured. The data showed a significant increase in plasma Vp after KSD179019 and secretin (positive control), but not in ACSF control (FIG. 10).

### Example 7: Anti-Hypertensive Effect of Secretin

Secretin is shown to trigger blood pressure reduction after its administration on rats [54]. We have provided evidence that intraperitoneal injection of secretin acutely reduce blood pressure of mice (FIG. 11A). In addition, this blood pressure drop was significant at 5 min post administration of the peptide (FIG. 11B). Further, secretin knockout mice (SCT^{-/-}) have significantly increased systolic, diastolic, and mean arterial blood pressure (FIG. 14).

### Example 8: Anti-Hypertensive Effect of KSD179019

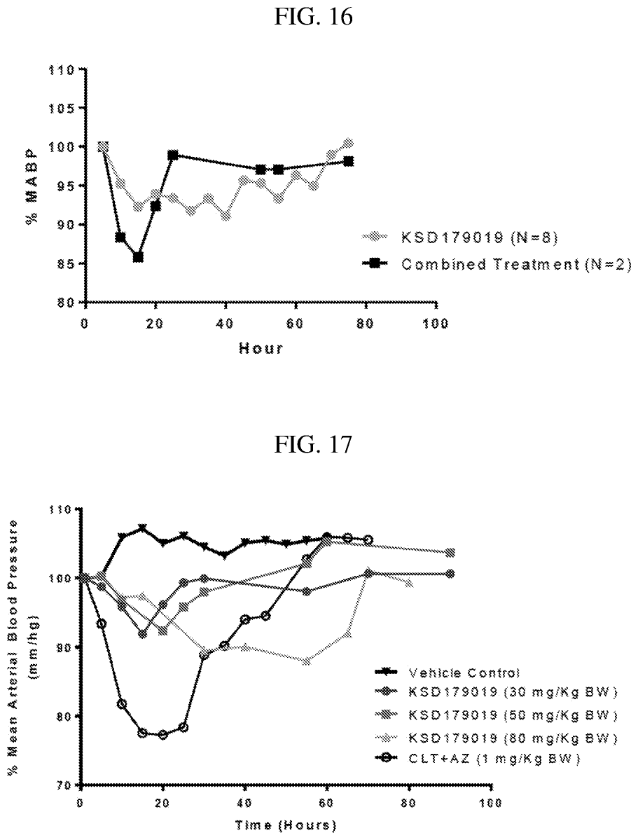

We tested the hypotensive effect of KSD179019 on spontaneously hypertensive (SHR) rats, a well-accepted animal model for hypertension [55]. We have administered 0.26 μg/g BW of the drug via tail vein injection and observed consistent blood pressure reduction (FIG. 12A), whereas, no reduction on the blood pressure was observed in saline control (FIG. 12B). The data indicated that reduction in blood pressure by KSD171990 lasted significantly up to 20 hours (FIG. 12C). Further, statistically significant drops in diastolic and systolic blood pressure from 20 to 50 hours post injection were observed (FIG. 16 and FIG. 17).

### Example 9: KSD179019 Comparison with Conventional Anti-Hypertensive Drugs

The hypotensive action of KSD179019 was compared with the conventional combination therapy Az+CLT (0.3 μg/g BW+1 μg/g BW) [53] (FIG. 13A) on SHR. Similar to published data, Az+CLT led to an initial drastic drop in the blood pressure of about 25% within 20 hours, but this drop of blood pressure was completely recovered to normal values after 25 hours post-injection. Whereas, KSD179019 possess a much more gentle and yet longer (10% drop peaked at 40 hours) hypotensive effect, which was observed even at 60 hours post-injection (FIG. 13B and FIG. 17).

### Example 10: Toxicity and Carcinogenicity Studies

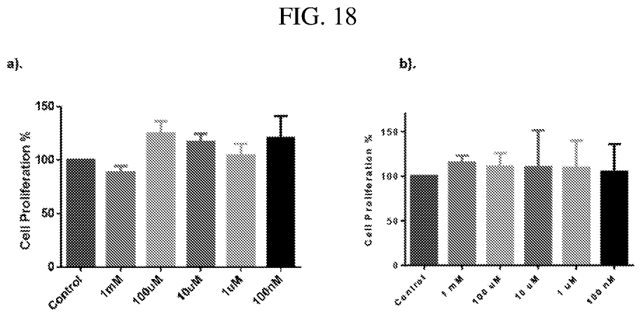

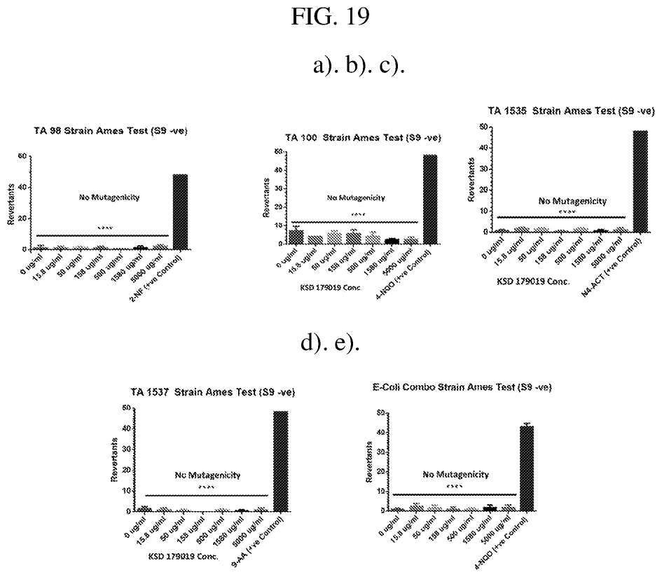

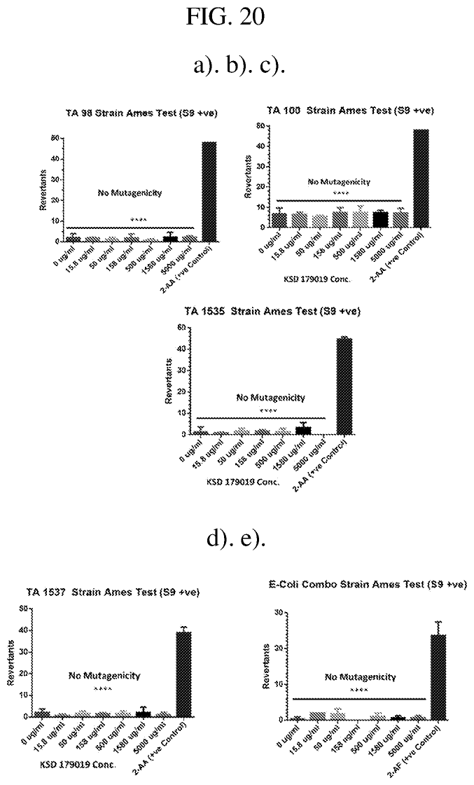

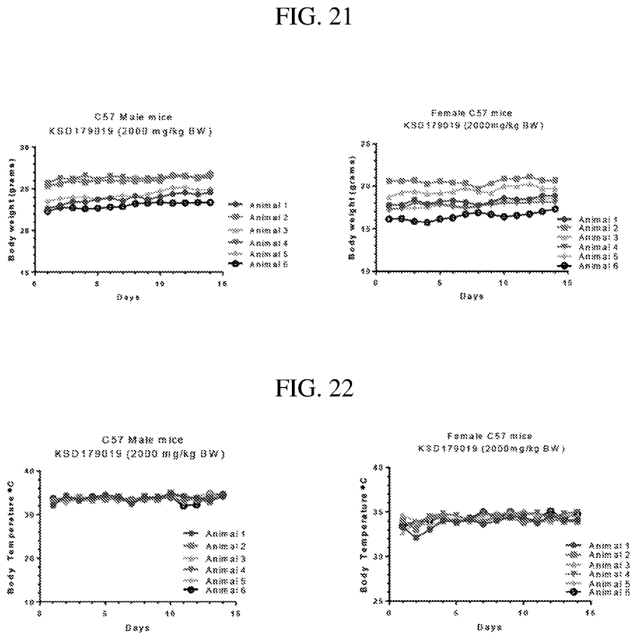

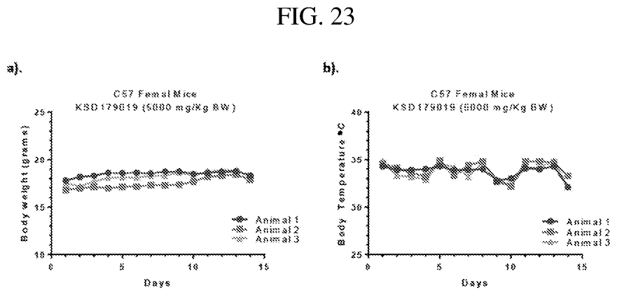

In vitro and in vivo toxicity testing was performed. All tests were performed using the OECD guidelines. For in vitro testing, KSD179019 organ/cell specific toxicity was determined using a cell viability testing (i.e. MTT assay). The MTT assay was performed on liver cells (HepG2 cell line) and kidney cells (HEK293 cell line) as they are the major organs effected by drug toxicity. It was observed that KSD179019 has no toxic effect on these cells at dose ranging from 100 nM to 1 mM (FIG. 18a; 18b). Additionally, the potential carcinogenicity of this drug is tested according to OECD-471 (Ames test) using the commercially available kit from XENOMETRIX. The mutation effect of the drug was tested in salmonella strains (TA98, TA100, TA1535 and TA1537) and on the combination of two E.coli strains (uvrA and pKM101) (FIGS. 19a; 19b; 19c; 19d and 19e). No mutational effect of KSD171990 was observed after the testing. Additionally, as the drug's secondary metabolite can also have carcinogenic effect, the Ames test was performed in presence of liver enzymes which can cause the formation of secondary metabolites (S9 liver extract) (FIGS. 20a; 20b; 20c; 20d and 20e). The results clearly show that there is no carcinogenic potential of KSD179019 or its metabolites. Further, in vivo toxicological test of KSD179019 were performed. The animal testing was done under an animal license from the Laboratory Animal Unit (LAU) of the University of Hong Kong for testing at acute, sub-chronic and chronic levels of exposure. The study was done based on the OECD-423 guidelines. The acute toxicity of KSD179019 was performed with single oral administration 2000 mg/kg BW (i.e. highest dose recommended by OECD to be tested for LD50 estimation) drug to estimate the Lethal Dose 50 (LD50) for the drug. After the KSD179019 administration, the animals were observed for food intake, water intake, digestion/constipation, urination, body temperature, change in skin and fur, drowsiness, aggressiveness, sleep, rate of respiration/breathing, eye color, ears, diarrhea, heartbeat, activity, general physique, coma, injury, hematochezia (blood stool), loss of appetite, convulsions, salivation, body weight, general reflex (CNS/ANS) and death (FIG. 10). In the course of the investigative period of 14 day after single dose of 2000 mg/kg BW, KSD179019 showed no effect on the overall health of animals. The test was done on 6 animals in two batches in both males and females. The body weight change remained under 2 grams (FIG. 21) which is normal growth for 9 weeks old C57BL/6J (https://www.jax.org/jax-mice-and-services/strain-data-sheet-pages/body-weight-chart-000664#) [66]. The body temperature (FIG. 22) remained constant for all the animals under observation. Furthermore, since no animal fatality was observed, the LD50 was estimated to be between 2000-5000 mg/kg BW. The animals were subjected to histological analysis post sacrificing on day 14. Major organs like brain, heart, liver, kidney, intestine, and colon were collected and analysed. None of these organs were observed to have any signs of necrosis. This observation indicated that the compound KSD179019 was well tolerated in the rodents. However, in order to determine a dose for sub-chronic and chronic studies, according to OECD recommendations, 3 animals were administered 5000 mg/kg BW of drug in a single administration. The test was performed on both male and female C57BL/6J mice and no fatal or toxic effects were observed. During the 14 day observation the animals remained healthy with no change in body weight and body temperature (FIG. 23).

### Example 11: Pharmacokinetic Studies

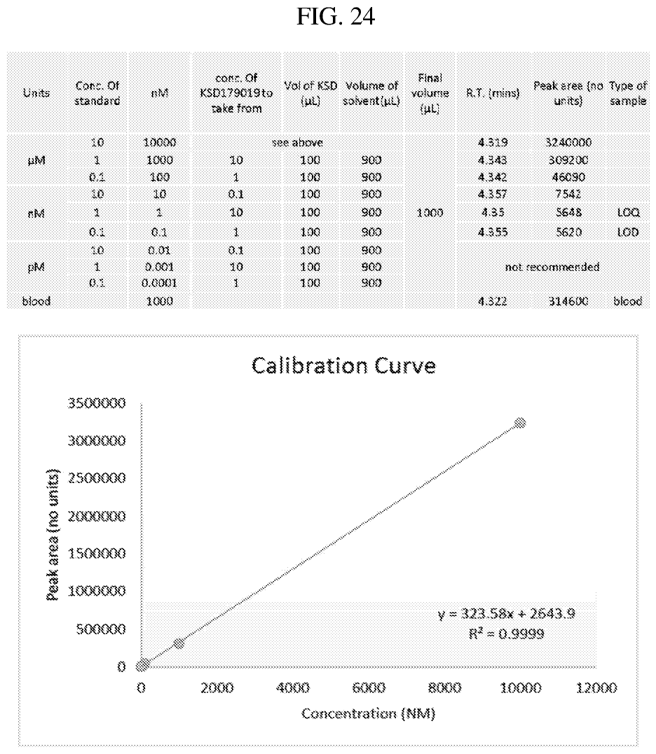

Working towards the pharmacokinetic studies, a standard curve for the KSD179019 on the LCMS/MS using MRM on Q-TOF was established (FIG. 24). This standard curve is used to develop and estimate the concentration of KSD179019 in blood and tissue samples to estimate the half-life and drug distribution study.

### Example 12: Design of Functional Analogs of KSD179019

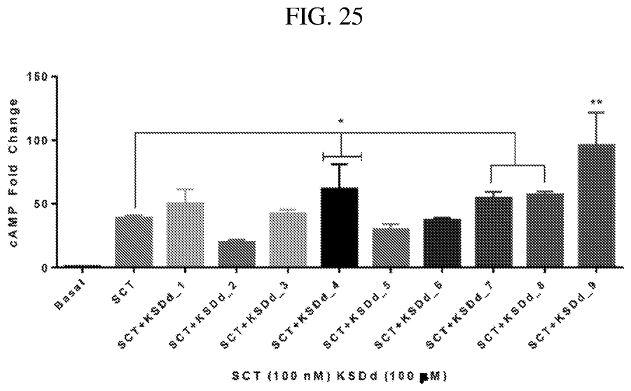

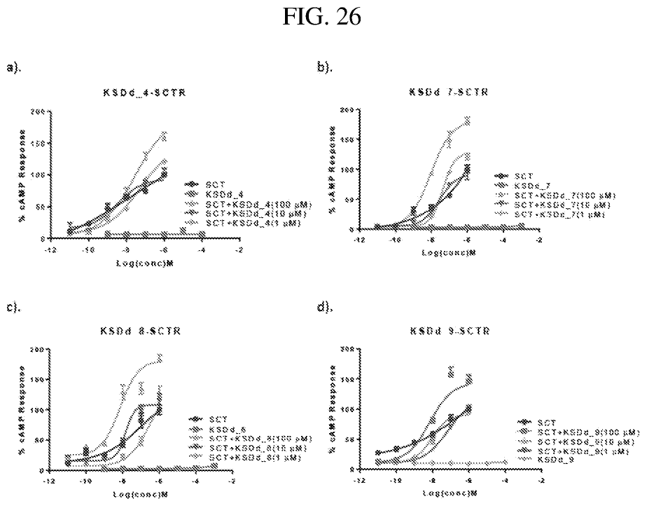

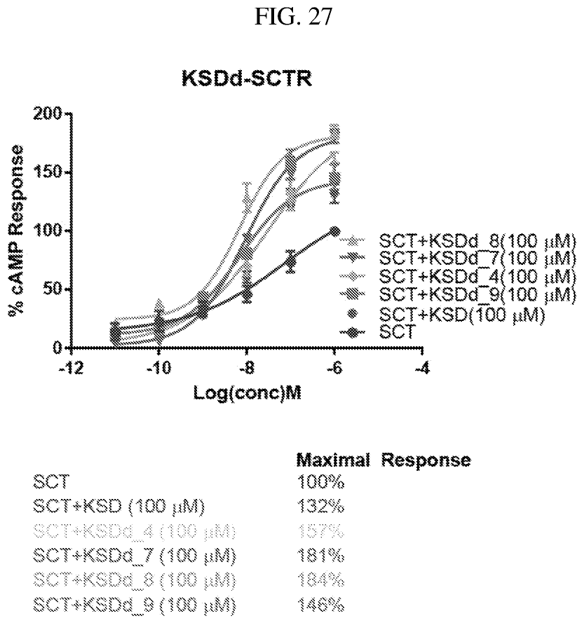

Functional analogs of KSD179019 were designed using an in silico technology. Initially, 9 derivatives of KSD179019 were developed and commercially synthesized under a CRO. These derivatives (KSDd) were tested in vitro with a secretin receptor activation assay via quantifying the secondary messenger in CHO cell system (FIG. 25). Four derivatives were identified (i.e. KSDd7, KSDd7, KSDd8, and KSDd9) to be functional as Positive Allosteric Modulators (PAM). Considering the maximal response by secretin as 100%, it was determined, for example, that KSDd4 was able to potentiate receptor activation by 157% (FIG. 26a). Whereas, the maximal response of KSDd7, KSDd8 and KSDd9 were 181%, 184% and 146% respectively compared to the secretin peptide (FIGS. 26b, 26c, and 26d). These results indicate that we generated several functionally active derivatives of KSD179019 which can be used in future should any toxic or other issue arises with KSD179019 itself. In the comparative in vitro analysis of KSD179019 derivatives (i.e. KSDd_4, KSDd_7, KSDd_8 and KSDd_9) with KSD179019, it was determined that KSDd_7 and KSDd_8 were the most potent derivatives followed by KSDd_4 and KSDd_9 (FIG. 27).

### Example 13: Summary

Close to 1 billion people are affected by hypertension worldwide and is estimated to increase to 1.56 billion individuals by 2025 [56]. Hypertension is an important risk factor for heart and cerebrovascular diseases since it can increase the risk of coronary heart disease, stroke and congestive heart failure by 3-, 7- and 6-folds, respectively [57]. Globally, hypertension-related deaths were documented to be 7.6 million in the year 2010 [58]. Conventionally, physicians start the therapy with a single drug at a low dose and the dose is escalated over time with constant observation of the patients for a drop in the blood pressure. This process involves continuous trial and error and hence many patients simply give up on the pills. The new approach of fixed-dose combination medications or “polypills” have been developed in recent times to avoid such issues [59]. Polypills are predeveloped pills with a definite ratio of conventional drugs and are in clinical trials [60]. The polypills have demonstrated the positive effect in 70% of the total population which is better than the conventional methodology with only 50% success. However, still, this approach remains limited to affect only 70% of the population [61]. Moreover, 30% of hypertension cases are deemed not affected even from the combination therapy, a condition known as resistant hypertension. Since resistant hypertension is currently untreatable by modern medication, it provides a niche for the novel drug development capable of treating such complications. At present, the best option for the treatment of hypertension has been, combination therapy i.e. modulating the blood pressure via multiple pathways [62]. Therefore, an unmet demand of development of novel drug candidate capable of modulating the blood pressure through two or more different pathways is required.

A decade of research, led by Professor B.K.C. Chow has established SCTR as novel, anti-hypertensive drug target. As SCTR is able to modulate several important physiological pathways therefore, secretin can cause blood pressure reduction by several molecular pathways. The primary anti-hypertensive effect of secretin is possibly due to its diuretic action, through its role in water homeostasis. Other potential pathways may include modulating the portal venous blood flow [63] and its vasodilatory action [64]. Evidently, recently developed synthetic secretin peptide analogs (SecreFlo™ [65]; ChiRhoStim® www.human-secretin.com) were reported to have mild bradycardia (reduced heart rate) and hypotension as side effects during clinical trials. Although, utilization of these peptides as therapeutics has remained restricted because of their short half-life in circulation (i.e. secretin-2 min, SecreFlo-20 min and ChiRhoStim-45 min). Also, high production and transportation expenses make peptide-based drugs difficult to be commercialized. Moreover, peptides based drugs has to be injected, to ensure absorption and inhibiting them from degradation in the digestive track. These limitations can be inhibited by developing small compound based drugs.

For the first time, the inventors have developed a small compound modulator for SCTR i.e. KSD179019. KSD179019 acts as a positive allosteric modulator therefore, it binds to a unique site on SCTR as compared to a direct agonist and exhibits minimal side effects. The inventors have demonstrated the in vivo anti-hypertensive action of KSD179019 in a hypertensive rat model i.e. SHR. Intravenous injection of KSD179019 (0.26 μg/g BW) significantly reduced the systolic and diastolic blood pressure (approximately 10% reduction in mean arterial blood pressure) of SHR for up to 60 hours post administration. Whereas, the intravenous injection of secretin peptide can reduce blood pressure for about only 10 min, which is consistent with its half-life in circulation, suggesting a much longer half-life of KSD179019. KSD179019 produced a much longer (360 times) hypotensive effect on SHR rats when compared to a secretin peptide and even 3 times longer than the conventional combination therapy (Az and CLT). A 60-hour effective window allows patients to take a single dose instead of thrice a day, which should improve patient's adherence to the regime. Additionally, the production cost of KSD179019 (available 17 USD/mg from targetmol.com) is about 13 times cheaper to that of secretin peptide (i.e. 220 USD/mg from an aspec.com). This difference will be inflated upon a large scale production of the drug. Furthermore, being lower than 900 Daltons, KSD179019 (i.e. 520.637 Dalton) should rapidly diffuse across cell membrane allowing transcellular transport through intestinal epithelial cells. These conditions are consistent according to “Rule of five” for a good oral small molecule drug candidate.

Advantageously, the animal studies demonstrated that KSD179019 had no acute toxicity and no carcinogenic effects. Henceforth, it can be concluded, that KSD179019 is a novel class of anti-hypertensive drug capable of dealing with the multifactorial disorder via several physiological pathways.

Furthermore, derivatives of KSD179019 were developed and tested, some of which derivatives have higher effects as positive allosteric modulators than KSD179019. These derivatives The use of this compound as a therapeutic not only reduces the drug load of combination therapy but also decreases the frequency of the drug regime. Moreover, the development of this new class of anti-hypertensive drug can also benefit patients suffering from resistant hypertension.

### EXEMPLARY EMBODIMENTS

Embodiment 1. A composition comprising KSD179019, or a derivative thereof, for use in a method for the treatment of hypertension, characterized in that the composition is administered to a subject in need of hypertension treatment in a therapeutically effective amount of the composition comprising KSD179019, or a derivative thereof, or a pharmaceutically acceptable salt thereof.

Embodiment 2. The composition according to Embodiment 1, wherein the composition further comprises a diuretic, beta-blocker, ACE inhibitor, angiotensin II receptor blocker, calcium channel blocker, alpha blocker, alpha-2 receptor antagonist, central agonist, peripheral adrenergic inhibitor, or vasodilator.

Embodiment 3. A method for making a KSD179019 analog and/or derivative, the method comprising: providing a three-dimensional (3D) model of a human secretin-bound secretin receptor (SCTR); virtually docking KSD179019 into the 3D model of the human secretin-bound SCTR using binding energy estimation; selecting at least one portion of KSD179019 that based on the virtual docking is located in close proximity to at least one amino acid of the KSD179019 docking site on the secretin-bound SCTR; introducing at least one molecular modification into the at least one selected portion of KSD179019 to generate a KSD179019 analog and/or derivative; virtually docking the KSD179019 analog and/or derivative into the docking site using binding energy estimation; and chemically manufacturing the KSD179019 analog and/or derivative provided that the KSD179019 analog and/or derivative virtually docks into the docking site with a binding energy estimate that is lower than the binding energy estimate of KSD179019.

Embodiment 4. The method according to Embodiment 3, wherein the KSD179019 analog and/or derivative has a same property and/or function as KSD179019.

Embodiment 5. The method according to Embodiment 3, wherein the KSD179019 analog and/or derivative has enhanced binding interaction with the secretin-secretin receptor complex compared to KSD179019.

Embodiment 6. The method according to Embodiment 4, wherein the KSD179019 analog and/or derivative has reduced binding interactions with non-SCTR receptors compared to KSD179019.

Embodiment 7. A composition comprising KSD179019, or a derivative thereof, for use in a method for the treatment of hypertension, characterized in that the composition is administered to a subject in need of hypertension treatment a therapeutically effective amount of the composition comprising a KSD179019 analog or derivative or a pharmaceutically acceptable salt thereof.

Embodiment 8. A composition comprising KSD179019, or a derivative thereof, for use in a method for the treatment of a condition or disease selected from the group consisting of gastritis, gastric acidity, gastrointestinal ulcer, pancreatitis and related disorders, liver cirrhosis, hepatoma, asthma, and bronchitis, characterized in that the composition is administered to a subject in need of hypertension treatment a therapeutically effective amount of the composition comprising KSD179019 and/or a KSD179019 analog and/or KSD179019 derivative or a pharmaceutically acceptable salt thereof.

Embodiment 9. A KSD179019 analog and/or derivative generated by the method of Embodiment 3.

Embodiment 10. A composition comprising the KSD179019 analog and/or derivative according to Embodiment 9 and an excipient.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Yaxley, J. P. and S. V. Thambar, Resistant hypertension: an approach to management in primary care. J Family Med Prim Care, 2015. 4(2): p. 193-9.
2. Adeloye, D. and C. Basquill, Estimating the prevalence and awareness rates of hypertension in Africa: a systematic analysis. PLoS One, 2014. 9(8): p. e104300.
3. Wang, J., et al., Prevalence, awareness, treatment, and control of hypertension in China: results from a national survey. Am J Hypertens, 2014. 27(11): p. 1355-61.
4. Zhai, Z., et al., Pulmonary hypertension in China: pulmonary vascular disease: the global perspective. Chest, 2010. 137(6 Suppl): p. 69S-77S.
5. Chu, J. Y., et al., Secretin as a neurohypophysial factor regulating body water homeostasis. Proc Natl Acad Sci USA, 2009. 106(37): p. 15961-6.
6. Bai, J. and B. K. Chow, Secretin is involved in sodium conservation through the renin-angiotensin-aldosterone system. FASEB J, 2017. 31(4): p. 1689-1697.
7. Lee, L. T., et al., Transmembrane peptides as unique tools to demonstrate the in vivo action of a cross-class GPCR heterocomplex. FASEB J, 2014. 28(6): p. 2632-44.
8. Bai, J. J., C. D. Tan, and B. K. C. Chow, Secretin, at the hub of water-salt homeostasis. Am J Physiol Renal Physiol, 2017. 312(5): p. F852-F860.
9. Cheng, C. Y., J. Y. Chu, and B. K. Chow, Vasopressin-independent mechanisms in controlling water homeostasis. J Mol Endocrinol, 2009. 43(3): p. 81-92.
10. Chu, J. Y. S., et al., Phenotypes developed in secretin receptor-null mice indicated a role for secretin in regulating renal water reabsorption. Molecular and Cellular Biology, 2007. 27(7): p. 2499.
11. Chu, J. Y. S., W. H. Yung, and B. K. C. Chow, Actions of secretin on the hypothalamic magnocellular neurons: Implications for the control of body water homostasis, in Regulatory Peptides. 2006, Elsevier BV. The Journal's web site is located at http://www.elsevier.com/locate/regpep. p. 159.
12. Funakoshi, A., K. Miyazaki, and H. Nawata, Effect of secretin and caerulein on pancreatic polypeptide and on insulin secretion from the isolated perfused ventral area of the rat pancreas. Regul Pept, 1989. 24(1): p. 111-6.
13. Glaser, B., et al., Effects of secretin on the normal and pathological beta-cell. J Clin
Endocrinol Metab, 1988. 66(6): p. 1138-43.
14. Sun, C., et al., Solution structure and mutational analysis of pituitary adenylate cyclase-activating polypeptide binding to the extracellular domain of PAC1-RS. Proc Natl Acad Sci USA, 2007. 104(19): p. 7875-80.
15. Grossini, E., et al., Intracoronary secretin increases cardiac perfusion and function in anaesthetized pigs through pathways involving beta-adrenoceptors and nitric oxide. Exp Physiol, 2013. 98(5): p. 973-87.
16. Gunnes, P., et al., Cardiovascular effects of secretin infusion in man. Scand J Clin Lab Invest, 1983. 43(7): p. 637-42.
17. Waldum, H. L., et al., The diuretic effect of secretin in man. Scand J Clin Lab Invest, 1980. 40(4): p. 381-7.
18. Chu, J. Y., et al., Secretin and body fluid homeostasis. Kidney Int, 2011. 79(3): p. 280-7.
19. Charlton, C. G., et al., Secretin receptors in the rat kidney: adenylate cyclase activation and renal effects. Peptides, 1986. 7(5): p. 865-71.
20. Virgolini, I., et al., Vasoactive intestinal peptide-receptor imaging for the localization of intestinal adenocarcinomas and endocrine tumors. N Engl J Med, 1994. 331(17): p. 1116-21
21. Abdel-Magid, A. F., Allosteric modulators: an emerging concept in drug discovery. ACS Med Chem Lett, 2015. 6(2): p. 104-7.
22. Henn, R. M., et al., Experience with synthetic secretin in the treatment of duodenal ulcer. Am J Dig Dis, 1976. 21(11): p. 921-5.
23. Shimizu, K., et al., The mechanism of inhibitory action of secretin on gastric acid secretion in conscious rats. J Physiol, 1995. 488 (Pt 2): p. 501-8.
24. Chey, W. Y. and T. M. Chang, Secretin: historical perspective and current status. Pancreas, 2014. 43(2): p. 162-82.
25. Gardner, T. B., E. D. Purich, and S. R. Gordon, Pancreatic duct compliance after secretin stimulation: a novel endoscopic ultrasound diagnostic tool for chronic pancreatitis. Pancreas, 2012. 41(2): p. 290-4.
26. Stevens, T. and M. A. Parsi, Update on endoscopic pancreatic function testing. World J Gastroenterol, 2011. 17(35): p. 3957-61.
27. Marolf, A. J., et al., Magnetic resonance (MR) imaging and MR cholangiopancreatography findings in cats with cholangitis and pancreatitis. J Feline Med Surg, 2013. 15(4): p. 285-94.
28. Lechin, F., B. Dijs, and B. Pardey-Maldonado, Insulin versus glucagon crosstalk: central plus peripheral mechanisms. Am J Ther, 2013. 20(4): p. 349-62.
29. LeSage, G., S. Glaser, and G. Alpini, Regulatory mechanisms of ductal bile secretion. Dig Liver Dis, 2000. 32(7): p. 563-6.
30. Farouk, M., et al., Secretin receptors in a new preparation of plasma membranes from intrahepatic biliary epithelium. J Surg Res, 1993. 54(1): p. 1-6.
31. Fukumoto, Y., et al., A new therapeutic trial of secretin in the treatment of intrahepatic cholestasis. Gastroenterol Jpn, 1989. 24(3): p. 298-307.
32. Alpini, G., et al., Upregulation of secretin receptor gene expression in rat cholangiocytes after bile duct ligation. Am J Physiol, 1994. 266(5 Pt 1): p. G922-8.
33. Rogers, D. F., Physiology of airway mucus secretion and pathophysiology of hypersecretion. Respir Care, 2007. 52(9): p. 1134-46; discussion 1146-9.
34. Davis, R. J., et al., Expression and functions of the duodenal peptide secretin and its receptor in human lung. Am J Respir Cell Mol Biol, 2004. 31(3): p. 302-8.
35. Hisatomi, A. and R. H. Unger, Secretin inhibits glucagon in the isolated perfused dog pancreas. Diabetes, 1983. 32(10): p. 970-3.
36. Boyns, D. R., R. J. Jarrett, and H. Keen, Intestinal hormones and plasma insulin: an insulinotropic action of secretin. Br Med J, 1967. 2(5553): p. 676-8.
37. Dupre, J., An Intestinal Hormone Affecting Glucose Disposal in Man. Lancet, 1964. 2(7361): p. 672-3.
38. Bainbridge, F. A. and A. P. Beddard, Secretin in Relation to Diabetes Mellitus. Biochem J, 1906. 1(8-9): p. 429-45.
39. Unger, R. H., et al., The effects of secretin, pancreozymin, and gastrin on insulin and glucagon secretion in anesthetized dogs. J Clin Invest, 1967. 46(4): p. 630-45.
40. Sekar, R. and B. K. Chow, Metabolic effects of secretin. General and comparative endocrinology, 2013. 181: p. 18-24.
41. Singh, K., et al., Structure-Activity Relationship Studies of N- and C-Terminally Modified Secretin Analogs for the Human Secretin Receptor. PLoS One, 2016. 11(3): p. e0149359.
42. Sunseri, J. and D. R. Koes, Pharmit: interactive exploration of chemical space. Nucleic Acids Res, 2016. 44(W1): p. W442-8.
43. Beinborn, M., Class B GPCRs: a hidden agonist within? Mol Pharmacol, 2006. 70(1): p. 1-4.
44. Hsu, K.-C., et al., iGEMDOCK: a graphical environment of enhancing GEMDOCK using pharmacological interactions and post-screening analysis. BMC Bioinformatics, 2011. 12(1): p. S33.
45. Trott, O. and A. J. Olson, AutoDock Vina: improving the speed and accuracy of docking with a new scoring function, efficient optimization, and multithreading. J Comput
Chem, 2010. 31(2): p. 455-61.
46. Dallakyan, S. and A. J. Olson, Small-molecule library screening by docking with PyRx. Methods Mol Biol, 2015. 1263: p. 243-50.
47. Mashiach, E., et al., FireDock: a web server for fast interaction refinement in molecular docking. Nucleic Acids Res, 2008. 36(Web Server issue): p. W229-32.
48. On, J. S., et al., Functional Pairing of Class B1 Ligand-GPCR in Cephalochordate Provides Evidence of the Origin of PTH and PACAP/Glucagon Receptor Family. Mol Biol Evol, 2015. 32(8): p. 2048-59.
49. Lee, V. H., et al., An indispensable role of secretin in mediating the osmoregulatory functions of angiotensin II. FASEB J, 2010. 24(12): p. 5024-32.
50. Cheng, C. Y., J. Y. Chu, and B. K. Chow, Central and peripheral administration of secretin inhibits food intake in mice through the activation of the melanocortin system. Neuropsychopharmacology, 2011. 36(2): p. 459-71.
51. Singh, K., et al., Glycyrrhizic Acid Reduces Heart Rate and Blood Pressure by a Dual Mechanism. Molecules, 2016. 21(10).
52. Cheng, J. W., Azilsartan/chlorthalidone combination therapy for blood pressure control. Integr Blood Press Control, 2013. 6: p. 39-48.
53. Kumar Puttrevu, S., et al., Pharmacokinetic-pharmacodynamic modeling of the antihypertensive interaction between azilsartan medoxomil and chlorthalidone in spontaneously hypertensive rats. Naunyn Schmiedebergs Arch Pharmacol, 2017. 390(5): p. 457-470.
54. Lawler, J. E., et al., Blood pressure and heart rate responses to environmental stress in the spontaneously hypertensive rat. Physiol Behav, 1985. 34(6): p. 973-6.
55. H′Doubler, P. B., Jr., et al., Spontaneously hypertensive and Wistar Kyoto rats are genetically disparate. Lab Anim Sci, 1991. 41(5): p. 471-3.
56. Kearney, P. M., et al., Global burden of hypertension: analysis of worldwide data. Lancet, 2005. 365(9455): p. 217-23.
57. Messerli, F. H., Cardiovascular effects of obesity and hypertension. Lancet, 1982. 1(8282): p. 1165-8.
58. Mills, K. T., et al., Global Disparities of Hypertension Prevalence and Control: A Systematic Analysis of Population-Based Studies From 90 Countries. Circulation, 2016. 134(6): p. 441-50.
59. Cimmaruta, D., et al., Polypill, hypertension and medication adherence: The solution strategy? Int J Cardiol, 2018. 252: p. 181-186.
60. Chrysant, S. G. and G. S. Chrysant, Usefulness of the Polypill for the Prevention of Cardiovascular Disease and Hypertension. Curr Hypertens Rep, 2016. 18(2): p. 14.
61. Lafeber, M., et al., Multifactorial Prevention of Cardiovascular Disease in Patients with Hypertension: the Cardiovascular Polypill. Curr Hypertens Rep, 2016. 18(5): p. 40.
62. Taddei, S., Combination therapy in hypertension: what are the best options according to clinical pharmacology principles and controlled clinical trial evidence? Am J Cardiovasc Drugs, 2015. 15(3): p. 185-94.
63. Bolondi, L., et al., Effect of secretin on portal venous flow. Gut, 1990. 31(11): p. 1306-10.
64. Ulrich, C. D., 2nd, M. Holtmann, and L. J. Miller, Secretin and vasoactive intestinal peptide receptors: members of a unique family of G protein-coupled receptors. Gastroenterology, 1998. 114(2): p. 382-97.
65. Secretin-repligen. SecreFlo. Drugs R D, 2002. 3(3): p. 217-9.
66. El Kabbaoui, M., et al., Acute and sub-chronic toxicity studies of the aqueous extract from leaves of Cistus ladaniferus L. in mice and rats. J Ethnopharmacol, 2017. 209: p. 147-156.

1. A composition comprising KSD179019, or a derivative thereof, for use in a method for the treatment of hypertension, characterized in that the composition is administered to a subject in need of hypertension treatment in a therapeutically effective amount of the composition comprising KSD179019, or a derivative thereof, or a pharmaceutically acceptable salt thereof.

2. The composition according to claim 1, wherein the composition further comprises a diuretic, beta-blocker, ACE inhibitor, angiotensin II receptor blocker, calcium channel blocker, alpha blocker, alpha-2 receptor antagonist, central agonist, peripheral adrenergic inhibitor, or vasodilator.

3. A method for making a KSD179019 analog and/or derivative, the method comprising: providing a three-dimensional (3D) model of a human secretin-bound secretin receptor (SCTR); virtually docking KSD179019 into the 3D model of the human secretin-bound SCTR using binding energy estimation; selecting at least one portion of KSD 179019 that based on the virtual docking is located in close proximity to at least one amino acid of the KSD179019 docking site on the secretin-bound SCTR; introducing at least one molecular modification into the at least one selected portion of KSD 179019 to generate a KSD 179019 analog and/or derivative; virtually docking the KSD179019 analog and/or derivative into the docking site using binding energy estimation; and chemically manufacturing the KSD179019 analog and/or derivative provided that the KSD 179019 analog and/or derivative virtually docks into the docking site with a binding energy estimate that is lower than the binding energy estimate of KSD179019.

4. The method according to claim 3, wherein the KSD179019 analog and/or derivative has a same property and/or function as KSD 179019.

5. The method according to claim 3, wherein the KSD179019 analog and/or derivative has enhanced binding interaction with the secretin-secretin receptor complex compared to KSD179019.

6. The method according to claim 4, wherein the KSD179019 analog and/or derivative has reduced binding interactions with non-SCTR receptors compared to KSD 179019.

7. A composition comprising KSD179019, or a derivative thereof, for use in a method for the treatment of hypertension, characterized in that the composition is administered to a subject in need of hypertension treatment a therapeutically effective amount of the composition comprising a KSD179019 analog or derivative or a pharmaceutically acceptable salt thereof.

8. A composition comprising KSD179019, or a derivative thereof, for use in a method for the treatment of a condition or disease selected from the group consisting of gastritis, gastric acidity, gastrointestinal ulcer, pancreatitis and related disorders, liver cirrhosis, hepatoma, asthma, and bronchitis, characterized in that the composition is administered to a subject in need of hypertension treatment a therapeutically effective amount of the composition comprising KSD179019 and/or a KSD179019 analog and/or KSD179019 derivative or a pharmaceutically acceptable salt thereof.

9. A KSD 179019 analog and/or derivative generated by the method of claim 3.

10. A composition comprising the KSD 179019 analog and/or derivative according to claim 9 and an excipient.
